SEQUENCE LISTING (13 sequences)

SEQ ID NO: 1 - DNA, 477 bp, Artificial Sequence
  Synthetic
atgtcctacgactacgagaaaaccagcctcaccctctaccgggcggtattcaaggccaactacgacggcgacgtcggtcgctacctgcatcccgacaaggaactcgccgaggctgcggaagtcgccccgctgctgcatccgaccttcgacagccccaacacccctggcgtccccgcccgcgcgccggacatcgtcgccggccgcgacggcctctacgccccggacaccggcggcacctcggtgttcgaccgcgccggcgtgctgcgccgcgccgacggcgacttcgtgatacccgacggcaccgacatcccgccggaccttaaggtgaagcaggacagctacaacaagcgcctgcaagccacccactacaccatcatgccggccaagccgatgtaccgggaggtcctcatgggccaactggacaacttcgtgcgcaacgccatccgccgccaatgggaaaaagcccgcgggctctag

SEQ ID NO: 2 - protein, 158 aa, Artificial Sequence
  Synthetic
MSYDYEKTSLTLYRAVFKANYDGDVGRYLHPDKELAEAAEVAPLLHPTFDSPNTPGVPARAPDIVAGRDGLYAPDTGGTSVFDRAGVLRRADGDFVIPDGTDIPPDLKVKQDSYNKRLQATHYTIMPAKPMYREVLMGQLDNFVRNAIRRQWEKARGL

SEQ ID NO: 3 - DNA, 477 bp, Artificial Sequence
  Synthetic
atgtcctacgactacgagaanaccagcctcaccctctaccgggcgntattcaaggccaactacganggcgangtcggtcgctacctgcnncccgacnaggaactcgccgaggcngcggaagtcgccccgctgctgcatccgaccttcgacagccccancaccccnggcgtccccgcccgcgcgccggacatcgtcgccggccgcgacggcctctacgccccggacaccggcggcacctcggtgttcgaccgcgccggcgtgctgcgccgcgccgacggcgacttcgtgatncccgacggcaccgacatcccgccggacctnaaggtgaancaggacagctacaacaagcgcctgcaagccacccactacaccatcatgccggccaagccgatgtaccgggaggtcctcatgggccanctggacaacttcgtgcgcaacgccatccgncgccaatgggaaaaagcccgcgggctctag

SEQ ID NO: 4 - protein, 246 aa, Artificial Sequence
  Synthetic
MSYDYEKTSLTLYRAXFKANYXGDVGRYLXPDKELAEAAEVAPLLHPTFDSPNTPGVPARAPDIVAGRDGLYAPDTGGTSVFDRAGVLRRADGDFVIPDGTDIPPDLKVKQDSYNKRLQATHYTIMPAKPMYREVLMGQLDNFVRNAIRRQWEKARGLLYAPDTGGTSVFDRAGVLRRADGDFVIPDGTDIPPDLKVKQDSYNKRLQATHYTIMPAKPMYREVLMGQLDNFVRNAIRRQWEKARGL

SEQ ID NO: 5 - protein, 149 aa, Artificial Sequence
  Synthetic
KTSLTLYRAVFKANYDGDVGRYLHPDKELAEAAEVAPLLHPTFDSPNTPGVPARAPDIVAGRDGLYAPDTGGTSVFDRAGVLRRADGDFVIPDGTDIPPDLKVKQDSYNKRLQATHYTIMPAKPMYREVLMGQLDNFVRNAIRRQWEKA

SEQ ID NO: 6 - protein, 237 aa, Artificial Sequence
  Synthetic
KTSLTLYRAXFKANYXGDVGRYLXPDKELAEAAEVAPLLHPTFDSPNTPGVPARAPDIVAGRDGLYAPDTGGTSVFDRAGVLRRADGDFVIPDGTDIPPDLKVKQDSYNKRLQATHYTIMPAKPMYREVLMGQLDNFVRNAIRRQWEKARGLLYAPDTGGTSVFDRAGVLRRADGDFVIPDGTDIPPDLKVKQDSYNKRLQATHYTIMPAKPMYREVLMGQLDNFVRNAIRRQWEKA

SEQ ID NO: 7 - protein, 158 aa, Artificial Sequence
  Synthetic
MSYDYEKTXXTLYRAXFKANYXGDVGRYLXPDKELAEAAEVAPLLHPTFDSPNTPGVPAXAPDIVAGRDGLYAPDTGGTSVFDRAGVLRRADGDFVIPDGTDIPPDLKVKQDSYNKRLXATHYTIMPAXXMYREVLMGXXDNFVRNAIXXQWEKARGL

SEQ ID NO: 8 - protein, 149 aa, Artificial Sequence
  Synthetic
KTXXTLYRAXFKANYXGDVGRYLXPDKELAEAAEVAPLLHPTFDSPNTPGVPAXAPDIVAGRDGLYAPDTGGTSVFDRAGVLRRADGDFVIPDGTDIPPDLKVKQDSYNKRLXATHYTIMPAXXMYREVLMGXXDNFVRNAIXXQWEKA

SEQ ID NO: 9 - DNA, 234 bp, Artificial Sequence
  Synthetic
atgaacctgaaaccccagaccctgatggtggcgatccagtgcgtcgccgcgcgcacccgcgaactcgacgcgcagttgcagaacgacgacccgcagaacgccgctgaactcgaacagttgctggtcggctacgacctcgccgccgacgacctgaagaacgcctacgagcaagccctgggccaatacagcggcctgccgccctatgaccggctgatcgaagagccggcatcctga

SEQ ID NO: 10 - protein, 77 aa, Artificial Sequence
  Synthetic
MNLKPQTLMVAIQCVAARTRELDAQLQNDDPQNAAELEQLLVGYDLAADDLKNAYEQALGQYSGLPPYDRLIEEPAS

SEQ ID NO: 11 - protein, 77 aa, Artificial Sequence
  Synthetic
MNLKPQTLMVAIQCVAARTRELDAQLQNDDPQNAAELEQLLVGYDLAADDLKNAYEXALGQYSGLPPYDRLIEEPAS

SEQ ID NO: 12 - DNA, 234 bp, Artificial Sequence
  Synthetic
atgaacctgaaaccccagaccctgatggtggcgatccagtgcgtcgccgcgcgcacccgcgaactcgacgcgcagttgcagaacgacgacccgcagaacgccgctgaactcgaacagntgctggtcggctacgacctcgccgccgacgacctgaagaacgcctacgannangccntggnncaatacagcggcctgccnccctatgaccggctgntcganganccggnnncctga

SEQ ID NO: 13 - protein, 61 aa, Artificial Sequence
  Synthetic
MNLKPQTLMVAIQCVAARTRELDAQLQNDDPQNAAELEQLLVGYDLAADDLKNAYEXALGQ

(19) United States
(12) Patent Application Publication
(10) Pub. No.: US 2012/0270271 A1
(43) Pub. Date: Oct. 25, 2012

(54) Toxin-Immunity System

(75) Inventor: Joseph Mougous, Seattle, WA (US)

(73) Assignee: UNIVERSITY OF WASHINGTON, Seattle, WA (US)

(21) Appl. No.: 13/516,158

(22) PCT Filed: Dec. 16, 2010
(86) PCT No.: PCT/US10/60873

§ 371 Date: Jul. 12, 2012

Related U.S. Application Data
(60) Provisional application No. 61/286,899, filed on Dec. 16, 2009.

(51) Int. Cl. C12N 15/64; C12N 1/21; C12N 5/10; C12N 1/13; C12N 1/19; C12N 15/63; C12P 19/34

(52) U.S. Cl. 435/91.1; 435/320.1; 435/252.33; 435/455; 435/468; 435/471; 435/257.2; 435/254.2; 435/348; 435/419; 435/325

(57) ABSTRACT

The present invention provides host cells whose survivability can be conditionally controlled, and vectors that can be used for preparing such host cells and for selectable cloning.

### CROSS REFERENCE

This application claims priority to U.S. Provisional Patent Application Ser. No. 61/286,899 filed Dec. 16, 2009, which is incorporated by reference herein in its entirety.

### STATEMENT OF U.S. GOVERNMENT INTEREST

This work was funded in part by NIH Grant No. AI080609, and the U.S. government has certain rights in the invention.

### BACKGROUND

Negative selection markers and their use in cloning vectors and cloning techniques are of great value in the field of molecular biology, particularly such vectors that can be used in any cell type.

Most genes in the literature that express a toxic protein, or “death genes”, are only functional in prokaryotic systems. Examples of such genes include rpsL, tetAR, pheS, thyA, lacY, gata-1, ccdB, and sacB. This invention disclosed herein provides an advantage of being active in both bacterial and eukaryotic cells, such that all embodiments disclosed herein can be utilized as would one of ordinary skill in the art in both cellular systems.

Antibiotic resistance genes are the most common selectable markers used in fermentation processes to avoid plasmid free cells to overgrow the culture. However antibiotics are expensive compounds and they, or their degradation products, can contaminate the biomass or production product. These contaminations are unacceptable from industrial, medical and regulatory perspectives. Consequently, when using antibiotics it has to be demonstrated that the final product is “antibiotic-free”. The assessment of the residual antibiotic levels and if necessary their removal are also costly procedures. Given these facts, the current trend in the industry is to forgo antibiotics in the production process altogether.

The increasing regulatory requirements to which biological agents are subjected will have a great impact in the field of industrial protein expression and production. There is an expectation that in a near future, there may be “zero tolerance” towards antibiotic-based selection and production systems. Besides the antibiotic itself, the antibiotic resistance gene is an important consideration. The complete absence of antibiotic-resistance gene being the only way to ensure that there is no propagation in the environment or transfer of resistance to pathogenic strains.

### SUMMARY OF THE INVENTION

In a first aspect, the present invention provides recombinant vectors, comprising a first gene coding for type VI secretion exported protein 2 (Tse2), wherein the first gene is operatively linked to a heterologous regulatory sequence.

In a second aspect, the present invention provides recombinant host cells comprising a recombinant vector according to any embodiment of the invention.

In a third aspect, the invention provides methods for selectable cloning, comprising culturing the recombinant host cell of any embodiment of the invention under conditions suitable for expression or disrupted expression of Tse2 from the recombinant vector if no insert is present, and selecting those cells that grow as comprising recombinant vectors with the insert cloned into the expression vector.

In a fourth aspect, the invention provides methods for producing a cloning vector that lacks an insert, comprising culturing the recombinant host cell of any embodiment of the invention under conditions suitable for vector replication and expression of Tse2, wherein the host cells further express a Tse2 antidote, and isolating vector from the host cells. In a further embodiment, the antidote comprises type VI secretion immunity protein 2 (Tsi2).

In a fifth aspect, the invention provides recombinant vectors, comprising a nucleic acid encoding Tsi2, wherein the nucleic acid is operatively linked to a regulatory sequence.

In a sixth aspect, the present invention provides recombinant host cells comprising the recombinant vector of any embodiment or combination of embodiments of the fifth aspect of the invention.

In a seventh aspect, the present invention provides host cells comprising in their genome, a first recombinant gene coding for type VI secretion exported protein 2 (Tse2) operatively linked to a regulatory sequence. In one embodiment, the host cells further comprise a second recombinant gene coding for an antidote for Tse2, wherein the second gene is operatively linked to a regulatory sequence. In one embodiment, the second recombinant gene coding the antidote may be episomal, such as in a plasmid or virus. In a further embodiment, the antidote comprises type VI secretion immunity protein 2 (Tsi2).

In an eighth aspect, the present invention relates to a kit comprising a carrier or receptacle being compartmentalized to receive and hold therein at least one container, wherein a first container contains linear or circular DNA molecule comprising a vector having at least one DNA fragment of the Tse2 gene sequence, as described herein. In another embodiment, the vector contained in the kit has at least one DNA fragment of the Tsi2 gene sequence, as described herein. In another embodiment, the kit contains one or more vectors which have at least one DNA fragment of the Tse2 sequence and vectors that have at least one DNA fragment of the Tsi2 sequence.

### BRIEF DESCRIPTION OF THE FIGURES

FIG. 1. Overview and results of an MS-based screen to identify H1-T6SS substrates. (A) Gene organization of P. aeruginosa HSI-I. Genes manipulated in this work are shown in color. (B) Activity of the H1-T6SS can be modulated by deletions of pppA and clpV1. Western blot analysis of Hcp1-V in the cell-associated (Cell) and concentrated supernatant (Sup) protein fractions from P. aeruginosa strains of specified genetic backgrounds. The genetic background for the parental strain is indicated below the blot. An antibody directed against RNA polymerase (-RNAP) is used as a loading control in this and subsequent blots. (C) Deletion of pppA causes increased p-Fha1-V levels. p-Fha1-V is observed by Western blot as one or more species with retarded electrophoretic mobility. (D) Spectral count ratio of C1 proteins detected in R1 and R2 of the comparative semi-quantitative secretome analysis of ΔpppA and ΔclpV1. The position of Hcp1 in both replicates is indicated. Proteins within the dashed line have SC ratios of <2-fold and constitute 89% of C1 proteins.

FIG. 2. Two VgrG-family proteins are regulated by retS and secreted in an H1-T6SS-dependent manner. (A) Overview of genetic loci encoding C2 proteins identified in R1 and R2 (green). RetS regulation of each ORF as determined by Goodman et al. is provided (Goodman et al., 2004). Genes not significantly regulated by RetS are filled grey. (B and C) Western blot analysis demonstrating that secretion of VgrG1-V (B) and VgrG4-V (C) is triggered in the ΔpppA background and is H1-T6SS (clpV1)-dependent. All blots are against the VSV-G epitope (-VSV-G).

FIG. 3. The Tse proteins are tightly regulated H1-T6SS substrates. (A) Tse secretion is under tight negative regulation by pppA and is H1-T6SS-dependent. Western analysis of Tse proteins expressed with C-terminal VSV-G epitope tag fusions from pPSV35 (Rietsch et al., 2005). Unless otherwise noted, all blots in this figure are -VSV-G. (B) H1-T6SS-dependent secretion of chromosomally-encoded Tse1-V measured by Western blot analysis. (C) Hcp1 secretion is independent of the tse genes. Western blot analysis of Hcp1 localization in control strains or strains lacking both vgrG1 and vgrG4, or the three tse genes. (D) The tse genes are not required for formation of a critical H1-T6S apparatus complex. Chromosomally-encoded ClpV1-GFP localization in the specified genetic backgrounds measured by fluorescence microscopy. TMA-DPH is a lipophilic dye used to visualize the position of cells. (E) The production and secretion of Tse proteins is dramatically increased in ΔretS. Western blot analysis of Tse levels from strains containing chromosomally-encoded Tse-VSV-G epitope tag fusions prepared in the wild-type or ΔretS backgrounds. Note—under conditions used to observe the high levels of Tse secretion in ΔretS, secretion cannot be visualized in ΔpppA as was demonstrated in (B).

FIG. 4. The Tse2 and Tsi2 proteins are a toxin-immunity module. (A) Tse2 is toxic to P. aeruginosa in the absence of Tsi2. Growth of the indicated P. aeruginosa strains containing either the vector control (−) or vector containing tse2 (+) under non-inducing (−IPTG) or inducing (+IPTG) conditions. (B) Tse2 and Tsi2 physically associate. Western blot analysis of samples before (Pre) and after (Post) -VSV-G immunoprecipitation from the indicated strain containing a plasmid expressing tsi2 (control) or tsi2-V. The glycogen synthase kinase (GSK) tag was used for detection of Tse2 (Garcia et al., 2006).

FIG. 5. Heterologously expressed Tse2 is toxic to prokaryotic and eukaryotic cells. (A) Tse2 is toxic to S. cerevisiae. Growth of S. cerevisiae cells containing a vector control or a vector expressing the indicated tse under non-inducing (Glucose) or inducing (Galactose) conditions. (B) Tsi2 blocks the toxicity of Tse2 in S. cerevisiae. Growth of S. cerevisiae harboring plasmids with the indicated gene(s), or empty plasmid(s), under non-inducing or inducing conditions. (C, D and E) Transfected Tse2 has a pronounced effect on mammalian cells. Flow cytometry (C) and fluorescence microscopy (D) analysis of GFP reporter co-transfection experiments with plasmids expressing the tse genes or tsi2. The percentage of rounded cells following the indicated transfections was determined (E) (n>500). Control (ctrl) experiments contained only the reporter plasmid. Bar graphs represent the average number from at least three independent experiments (±SEM). (F and G) Expression of tse2 inhibits the growth of E. coli (F) and B. thailandensis (G). E. coli (F) and B. thailandensis (G) were transformed with expression plasmids regulated by inducible expression with IPTG (F) or rhamnose (G), respectively, containing no insert, tse2, or both the tse2 and tsi2 loci. Growth on solid medium was imaged after one (F) or two (G) days of incubation.

FIG. 6. Immunity to Tse2 provides a growth advantage against P. aeruginosa strains secreting the toxin by the H1-T6SS. (A) Tse2 secreted by the H1-T6SS of P. aeruginosa does not promote cytotoxicity in HeLa cells. LDH release by HeLa cells following infection with the indicated P. aeruginosa strains or E. coli. P. aeruginosa strain PA14 and E. coli were included as highly cytotoxic and non-cytotoxic controls, respectively. Bars represent the mean of five independent experiments ±SEM. (B and C) Results of in vitro growth competition experiments in liquid medium (B) or on a solid support (B and C) between P. aeruginosa strains of the indicated genotypes. The parental strain is ΔretS. The ΔclpV1 and Δtsi2-dependent effects were complemented as indicated by +clpV1 and +tsi2, respectively (see methods). Bars represent the mean donor:recipent CFU ratio from three independent experiments (±SEM).

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

All embodiments of any aspect of the invention can be used in combination, unless the context clearly dictates otherwise.

In a first aspect, the present invention provides recombinant vectors, comprising a first gene coding for type VI secretion exported protein 2 (Tse2), wherein the first gene is operatively linked to a heterologous regulatory sequence. As shown in the examples that follow, intracellular Tse2 is toxic to a broad spectrum of prokaryotic and eukaryotic cells. Thus, Tse2 can be used, for example, in negative selection cloning in both prokaryotes and eukaryotes. Tse2 can also be used when selection using an antibiotic is not suitable to the experiment design. Use of this system can avoid trace antibiotics from remaining in the system.

As used herein, a “gene” is any nucleic acid capable of expressing the recited protein, and thus includes genomic DNA, mRNA, cDNA, etc.

As used herein, a “vector” can be a circular vector such as a lambda vector or a linearized vector such as a linearized plasmid or viral vector.

The invention also relates to vectors comprising one or more of the nucleic acid molecules used in the invention and/or used in methods of the invention. In accordance with the invention, any vector may be used to construct the vectors of invention. In particular, vectors known in the art and those commercially available (and variants or derivatives thereof) may in accordance with the invention be engineered to include one or more nucleic acid molecules encoding one or more recombination sites (or portions thereof), or mutants, fragments, or derivatives thereof, for use in the methods of the invention. Such vectors may be obtained from, for example, Vector Laboratories Inc.; Promega; Novagen; New England Biolabs; Clontech; Roche; Pharmacia; EpiCenter; OriGenes Technologies Inc.; Stratagene; Perkin Elmer; Pharmingen; and Invitrogen Corp., Carlsbad, Calif. Such vectors may then for example be used for cloning or subcloning nucleic acid molecules of interest. General classes of vectors of particular interest include prokaryotic and/or eukaryotic cloning vectors, Expression Vectors, fusion vectors, two-hybrid or reverse two-hybrid vectors, shuttle vectors for use in different hosts, mutagenesis vectors, transcription vectors, and the like.

Other vectors of interest include viral origin vectors (M13 vectors, bacterial phage .lamda. vectors, bacteriophage P1 vectors, adenovirus vectors, herpesvirus vectors, retrovirus vectors, phage display vectors, combinatorial library vectors), high, low, and adjustable copy number vectors, vectors which have compatible replicons for use in combination in a single host (pACYC184 and pBR322) and eukaryotic episomal replication vectors (pCDM8).

Particular vectors of interest include prokaryotic Expression Vectors such as pcDNA II, pSL301, pSE280, pSE380, pSE420, pTrcHisA, B, and C, pRSET A, B, and C (Invitrogen Corp., Carlsbad, Calif.), pGEMEX-1, and pGEMEX-2 (Promega, Inc.), the pET vectors (Novagen, Inc.), pTrc99A, pKK223-3, the pGEX vectors, pEZZ18, pRIT2T, and pMC1871 (Pharmacia, Inc.), pKK233-2 and pKK388-1 (Clontech, Inc.), and pProEx-HT (Invitrogen Corp., Carlsbad, Calif.) and variants and derivatives thereof. Destination Vectors can also be made from eukaryotic Expression Vectors such as pFastBac, pFastBac HT, pFastBac DUAL, pSFV, and pTet-Splice (Invitrogen Corp., Carlsbad, Calif.), pEUK-C1, pPUR, pMAM, pMAMneo, pBI101, pBI121, pDR2, pCMVEBNA, and pYACneo (Clontech), pSVK3, pSVL, pMSG, pCH110, and pKK232-8 (Pharmacia, Inc.), p3′SS, pXT1, pSG5, pPbac, pMbac, pMC1neo, and pOG44 (Stratagene, Inc.), and pYES2, pAC360, pBlueBacHis A, B, and C, pVL1392, pBsueBacIll, pCDM8, pcDNA1, pZeoSV, pcDNA3 pREP4, pCEP4, and pEBVHis (Invitrogen Corp., Carlsbad, Calif.) and variants or derivatives thereof.

Other vectors of particular interest include pUC18, pUC19, pBlueScript, pSPORT, cosmids, phagemids, YACs (yeast artificial chromosomes), BACs (bacterial artificial chromosomes), MACs (mammalian artificial chromosomes), pQE70, pQE60, pQE9 (Quiagen), pBS vectors, PhageScript vectors, BlueScript vectors, pNH8A, pNH16A, pNH18A, pNH46A (Stratagene), pcDNA3 (Invitrogen, Carlsbad, Calif.), pGEX, pTrsfus, pTrc99A, pET-5, pET-9, pKK223-3, pKK233-3, pDR540, pRIT5 (Pharmacia), pSPORT1, pSPORT2, pCMVSPORT2.0 and pSV-SPORT1 (Invitrogen Corp., Carlsbad, Calif.) and variants or derivatives thereof.

Additional vectors of interest include pTrxFus, pThioHis, pLEX, pTrcHis, pTrcHis2, pRSET, pBlueBacHis2, pcDNA3.1/His, pcDNA3.1(−)/Myc-His, pSecTag, pEBVHis, pPIC9K, pPIC3.5K, pAO815, pPICZ, pGAPZ, pBlueBac4.5, pBlueBacHis2, pMelBac, pSinRep5, pSinHis, pIND, pIND(SP1), pVgRXR, pcDNA2.1. pYES2, pZErO1.1, pZErO-2.1, pCR-Blunt, pSE280, pSE380, pSE420, pVL1392, pVL1393, pCDM8, pcDNA1.1, pcDNA1.1/Amp, pcDNA3.1, pcDNA3.1/Zeo, pSe, SV2, pRc/CMV2, pRc/RSV, pREP4, pREP7, pREP8, pREP9, pREP10, pCEP4, pEBVHis, pCR3.1, pCR2.1, pCR3.1-Uni, and pCRBac from Invitrogen; .lamda.gt11, pTrc99A, pKK223-3, pGEX-2T, pGEX-2TK, pGEX-4T-1, pGEX-4T-2, pGEX-4T-3, pGEX-3X, pGEX-5X-1, pGEX-5X-2, pGEX-5X-3, pEZZ18, pRIT2T, pMC1871, pSVK3, pSVL, pMSG, pCH110, pKK232-8, pSL1180, pNEO, and pUC4K from Pharmacia; pSCREEN-lb(+), pT7Blue(R), pT7Blue-2, pCITE-4-abc(+), pOCUS-2, pTAg, pET-32 LIC, pET-30 LIC, pBAC-2 cp LIC, pBACgus-2 cp LIC, pT7Blue-2 LIC, pT7Blue-2, pET-3abcd, pET-7abc, pET9abcd, pET11abcd, pET12abc, pET-14b, pET-15b, pET-16b, pET-17b-pET-17xb, pET-19b, pET-20b(+), pET-21abcd(+), pET-22b(+), pET-23abcd(+), pET-24abcd(+), pET-25b(+), pET-26b(+), pET-27b(+), pET-28abc(+), pET-29abc(+), pET-30abc(+), pET-31b(+), pET-32abc(+), pET-33b(+), pBAC-1, pBACgus-1, pBAC4x-1, pBACgus4x-1, pBAC-3 cp, pBACgus-2 cp, pBACsurf-1, plg, Signal plg, pYX, Selecta Vecta-Neo, Selecta Vecta-Hyg, and Selecta Vecta-Gpt from Novagen; pLexA, pB42AD, pG13T9, pAS2-1, pGAD424, pACT2, pGAD GL, pGAD GH, pGAD10, pGilda, pEZM3, pEGFP, pEGFP-1, pEGFP-N, pEGFP-C, pEBFP, pGFPuv, pGFP, p6xHis-GFP, pSEAP2-Basic, pSEAP2-Contral, pSEAP2-Promoter, pSEAP2-Enhancer, p.beta.gal-Basic, p.beta.gal-Control, p.beta.gal-Promoter, p.beta.gal-Enhancer, pTet-Off, pTet-On, pTK-Hyg, pRetro-Off, pRetro-On, pIRES1neo, pIRES1hyg, pLXSN, pLNCX, pLAPSN, pMAMneo, pMAMneo-CAT, pMAMneo-LUC, pPUR, pSV2neo, pYEX 4T-1/2/3, pYEX-S1, pBacPAK-His, pBacPAK8/9, pAcUW31, BacPAK6, pTriplEx, .lamda.gt10, .lamda.gt11, and pWE15, and from Clontech; Lambda ZAP II, pBK-CMV, pBK-RSV, pBluescript II KS +/−, pBluescript II SK +/−, pAD-GAL4, pBD-GAL4 Cam, pSurfscript, Lambda FIX II, Lambda DASH, Lambda EMBL3, Lambda EMBL4, SuperCos, pCR-Scrigt Amp, pCR-Script Cam, pCR-Script Direct, pBS +/−, pBC KS +/−, pBC SK +/−, Phagescript, pCAL-n-EK, pCAL-n, pCAL-c, pCAL-kc, pET-3abcd, pET-11abcd, pSPUTK, pESP-1, pCMVLacI, pOPRSVI/MCS, pOPI3 CAT, pXT1, pSG5, pPbac, pMbac, pMC1 neo, pMC1 neo Poly A, pOG44, p0045, pFRT.beta.GAL, pNEO.beta.GAL, pRS403, pRS404, pRS405, pRS406, pRS413, pRS414, pRS415, and pRS416 from Stratagene.

Two-hybrid and reverse two-hybrid vectors of particular interest include pPC86, pDBLeu, pDBTrp, pPC97, p2.5, pGAD1-3, pGAD10, pACt, pACT2, pGADGL, pGADGH, pAS2-1, pGAD424, pGBT8, pGBT9, pGAD-GAL4, pLexA, pBD-GAL4, pHISi, pHISi-1, placZi, pB42AD, pDG202, pJK202, pJG4-5, pNLexA, pYESTrp and variants or derivatives thereof.

Yeast Expression Vectors of particular interest include pESP-1, pESP-2, pESC-His, pESC-Trp, pESC-URA, pESC-Leu (Stratagene), pRS401, pRS402, pRS411, pRS412, pRS421, pRS422, and variants or derivatives thereof.

Vectors according to this aspect of the invention include, but are not limited to: pENTR1A, pENTR2B, pENTR3c, pENTR4, pENTR5, pENTR6, pENTR7, pENTR8, pENTR9, pENTR10, pENTR11, pDEST1, pDEST2, pDEST3, pDEST4, pDEST5, pDEST6, pDEST7, pDEST8, pDEST9, pDEST10, pDEST11, pDEST12.2 (also known as pDEST12), pDEST13, pDEST14, pDEST15, pDEST16, pDEST17, pDEST18, pDEST19, pDEST20, pDEST21, pDEST22, pDEST23, pDEST24, pDEST25, pDEST26, pDEST27, pEXP501 (also known as pCMVSPORT6.0), pDONR201, pDONR202, pDONR203, pDONR204, pDONR205, pDONR206, pDONR212, pDONR212(F) (FIGS. 28A-28C), pDONR212(R) (FIGS. 29A-29C), pMAB58, pMAB62, pDEST28, pDEST29, pDEST30, pDEST31, pDEST32, pDEST33, pDEST34, pDONR207, pMAB85, pMAB86, a number of which are described in PCT Publication WO 00/52027 (the entire disclosure of which is incorporated herein by reference), and fragments, mutants, variants, and derivatives of each of these vectors. However, it will be understood by one of ordinary skill that the present invention also encompasses other vectors not specifically designated herein, which comprise one or more of the isolated nucleic acid molecules used in the invention encoding one or more recombination sites or portions thereof (or mutants, fragments, variants or derivatives thereof), and which may further comprise one or more additional physical or functional nucleotide sequences described herein which may optionally be operably linked to the one or more nucleic acid molecules encoding one or more recombination sites or portions thereof. Such additional vectors may be produced by one of ordinary skill according to the guidance provided in the present specification.

As used herein, the term “cell” is referring to either a prokaryotic or a eukaryotic cell unless otherwise designated.

In one preferred embodiment, the first gene comprises or consists of a nucleotide sequence that encode a P. aeruginosa Tse2 amino acid sequence according to SEQ ID NO:2. In another preferred embodiment, the first gene comprises or consists of a nucleotide sequence according to SEQ ID NO:1.

Closely related Tse2 genes and Tse2 proteins are present in other P. aeruginosa strains, with variable positions noted in SEQ ID NOS:3-4. Thus, in another preferred embodiment, the first gene comprises or consists of a nucleotide sequence that can encode an amino acid sequence according to SEQ ID NO:4. In another preferred embodiment, the first gene comprises or consists of a nucleotide sequence according to SEQ ID NO:3.

As used herein, “Tse2” includes functional equivalents (truncations, mutants, etc.) thereof, wherein such equivalents maintain cytotoxic activity as described herein. Methods for identifying such functional equivalents are disclosed herein and a variety of such functional equivalents are disclosed. For example, the inventors have discovered that residues 1-6 and 156-158 of Tse2 are not required for toxicity (See Table 1 below). Thus, in another embodiment, the first gene comprises or consists of a nucleotide sequence that can encode an amino acid sequence according to SEQ ID NO:5 or SEQ ID NO:6.

The inventors have further identified a series of Tse2 mutant polypeptides that retain toxicity. Specifically, the inventors have shown (see below) that mutations at positions 9, 10, 60, 119, 129, 130, 139, 140, 149, and 150 of SEQ ID NO:2 can be tolerated while retaining toxicity (See Table 2 below). Thus, in another embodiment, the first gene encodes a mutant Tse2 polypeptide that differs from the amino acid sequence of SEQ ID NO:2 by an amino acid substitution at one or more of amino acid residues 9, 10, 60, 119, 129, 130, 139, 140, 149, and 150, and is optionally deleted for one or more of resides 1-6 and one or more of residues 156-158. In another embodiment, the first gene encodes a mutant Tse2 polypeptide that includes one or more amino acid substitutions selected from the group consisting of S9A. L10A, R60A, Q119A, K129A, P129A, Q139A, L139A, R149A, and R150A. In a further preferred embodiment, the first gene comprises or consists of a nucleotide sequence that can encode an amino acid sequence according to SEQ ID NO:7 or SEQ ID NO:8.

The regulatory sequence is “heterologous”, meaning that it is not a naturally occurring Tse2 regulatory region. As used herein, a “regulatory sequence” is any nucleic acid sequence that regulates or affects (i) transcription, (ii) translation, and/or (iii) post-translational modifications, during expression of a gene operatively linked the regulatory nucleic acid, and which contains one or more “control elements” for regulating such activity. The term “control element” of a regulatory nucleic acid is well known in the art (see, e.g., Goeddel, Gene Expression Technology, Methods in Enzymology 185, Academic Press, San Diego, Calif., 1990), and includes, e.g., transcriptional promoters, transcriptional enhancer elements, transcriptional termination signals, polyadenylation sequences (located 3′ to the translation stop codon), sequences for optimization of initiation of translation (located 5′ to the coding sequence), translation termination sequences, sequences that direct post-translational modification (e.g., glycosylation sites), all of which may be used to regulate the transcription and/or translation of a gene operatively linked to a regulatory sequence. It shall be appreciated by those skilled in the art that the selection of control elements of a regulatory nucleic acid will depend on such factors as the choice of the host cell to be transformed, the level of expression of protein desired, etc.

The term “promoter” includes any nucleic acid sequence sufficient to direct transcription in the host cell, including inducible promoters, repressible promoters and constitutive promoters. Exemplary promoters include bacterial, viral, algal, mammalian and yeast promoters, as are well known in the art. Many such promoters, including inducible promoters, are commercially available from vendors including Life Technologies, System Biosciences, and Promega Biosciences. Exemplary promoters for expression in E. coli include, but are not limited to lac, tip, ptrc, and T7 promoters. Exemplary promoters useful for expressing proteins in eukaryotic cells include but are not limited to the baculovirus polyhedrin, SP6, metallothionein I, Autographa californica nuclear polyhidrosis virus, Semliki Forest virus, Tet, CMV, Gall, Ga110, and T7 promoters.

In one embodiment, the Tse2 gene is operatively linked to a promoter element sufficient to render promoter-dependent controllable gene expression, for example, inducible or repressible by external signals or agents (adding/removing compounds from the growth media for the recombinant cells), or by altering culture conditions (temperature, pH, etc.). Exemplary controllable promoters are those that are alcohol-regulated, tetracycline-regulated, steroid-regulated, metal-regulated, pathogen-regulated, light-regulated, or temperature-regulated. For use in bacterial systems, many controllable promoters are known (Old and Primrose, 1994). Common examples include P_{lac} (IPTG), P_{tac} (IPTG), lambdaP_{R} (loss of CI repressor), lambdaP_{L} (loss of CI repressor), P_{trc} (IPTG), Pₜᵣₚ (IAA). The controlling agent is shown in brackets after each promoter. Examples of controllable plant promoters include the root-specific ANRI promoter (Zhang and Forde (1998) Science 279:407) and the photosynthetic organ-specific RBCS promoter (Khoudi et al. (1997) Gene 197:343). Further exemplary controllable promoters include the Tet-system (Gossen and Bujard, PNAS USA 89: 5547-5551, 1992), the ecdysone system (No et al., PNAS USA 93: 3346-3351, 1996), the progesterone-system (Wang et al., Nat. Biotech 15: 239-243, 1997), and the rapamycin-system (Ye et al., Science 283:88-91, 1999), arabinose-inducible promoters, and rhamnose-inducible promoters.

Expression vectors and methods for their engineering and isolation are well known in the art (see, e.g., Maniatis et al., supra), or they can be obtained through a commercial vendor, e.g., Invitrogen (Carlsbad, Calif.), Promega (Madison, Wis.), and Statagene (La Jolla, Calif.) and modified as needed. Examples of commercially available expression vectors include pcDNA3 (Invitrogen), Gateway cloning technology (Life Technologies), and pCMV-Script (Stratagene). Vector components, regulatory nucleic acids, etc. are typically available from a commercial source or can be isolated from a natural source (e.g., animal tissue or microorganism) or prepared using a synthetic means such as PCR. The arrangement of the components can be any arrangement practically desired by one of ordinary skill in the art. Vectors used in the present invention can be derived from viral genomes that yield virions or virus-like particles, which may or may not replicate independently as extrachromosomal elements. Virion particles can be introduced into the host cells by infection. The viral vector may become integrated into the cellular genome. Examples of viral vectors for transformation of mammalian cells are SV40 vectors, and vectors based on papillomavirus, adenovirus, Epstein-Barr virus, vaccinia virus, and retroviruses, such as Rous sarcoma virus, or a mouse leukemia virus, such as Moloney murine leukemia virus. For mammalian cells, electroporation or viral-mediated introduction can be used.

In one embodiment, the vector comprises one or more unique restriction enzyme recognition sites, wherein cloning of a nucleic acid insert into the one or more unique restriction enzyme recognition sites disrupts expression of Tse2. The vectors of this embodiment can be used as cloning vehicles, since cloning of an insert into the one or more restriction sites in the vector interrupts Tse2 expression and provide an easily selectable marker—cells with vectors containing no insert have their growth inhibited by Tse2 expression (so long as they do not endogenously express an antidote to Tse2), and those with inserts do not. In one preferred embodiment, one or more unique restriction sites are engineered into the coding region for Tse2 using techniques well known to those of skill in the art, such that cloning an insert into the restriction site disrupts the coding region for Tse2. In this embodiment, the restriction sites can be engineered into the coding region to result in silent nucleotide changes, or may result in one or more changes in the amino acid sequence of Tse2, so long as the encoded Tse2 protein retains cytotoxic activity. Alternatively, the one or more unique restriction sites may be located in regulatory regions such that cloning of an insert would disrupt expression of Tse2 from the vector. Design and synthesis of nucleic acid sequences and preparation of vectors comprising such sequences is well within the level of skill in the art.

The invention relates to a novel cloning and/or sequencing vector which includes at least one promoter nucleotide sequence and at least one nucleotide sequence encoding a fusion protein (Tse2) which is active as a poison, the said nucleotide sequence being obtained by fusing a gene coding nucleotide sequence which includes multiple unique cloning sites (MCS) and a nucleotide sequence which encodes Tse2. An analogous system utilizing the prokaryotic death gene ccdB has been described in U.S. Pat. No. 7,176,029, and is incorporated by reference herein in its entirety. Exemplary fusion protein partners to fuse with Tse 2 comprise, but are not limited to, lacZα, GFP, RFP, His, and FLAG.

In one non-limiting embodiment, the cloning vector contains the Tse2 gene fused to the C-terminus or N-terminus of LacZα. The expression of the Tse2-LacZ fusion protein is controlled by an inducible promoter, such as the lac promoter, such that expression of the Tse2-LacZ fusion protein will result in the death of a cell. In certain embodiments, a MCS is contained within the LacZ gene, such that insertion of a DNA fragment disrupts the expression of the lacZα-Tse2 gene fusion, permitting growth of only positive recombinants. Cells that contain nonrecombinant vector do not survive.

Plasmids according to this embodiment allow doubly digested restriction fragments to be cloned in both orientations with respect to the lac promoter. Insertion of a restriction fragment into one of the unique cloning sites interrupts the genetic information of the gene fusion, leading to the synthesis of a gene fusion product which is not functional. Insertional inactivation of the gene fusion ought always to take place when a termination codon is introduced or when a change is made in the reading frame. The cells which harbor a recombinant vector (disrupted Tse2) will be viable while cells which harbor an intact vector (intact Tse2) will not be viable. This negative selection, by simple culture on a solid medium, makes it possible to eliminate cells which harbor a non-recombinant vector (non-viable clones) and to select recombinant clones (viable clones).

In another embodiment, the recombinant vector comprises one or more recombination sites flanking the Tse2 gene. In a preferred embodiment, the recombinant vector comprises at least a first and a second recombination site flanking a first gene coding for Tse2 operatively linked to a regulatory sequence, wherein said first and second recombination sites do not recombine with each other. As used herein, a “recombination site” is a discrete section or segment of DNA that is recognized and bound by a site-specific recombination protein during the initial stages of integration or recombination. For example, the recombination site for Cre recombinase is loxP, a 34 base pair sequence comprised of two 13 base pair inverted repeats (serving as the recombinase binding sites) flanking an 8 base pair core sequence. See Sauer, B., Curr. Opin. Biotech. 5:521-527 (1994). Other examples of recognition sequences include the attB, attP, attL, and attR sequences which are recognized by the recombination protein lambda. attB is an approximately 25 base pair sequence containing two 9 base pair core-type Int binding sites and a 7 base pair overlap region, while attP is an approximately 240 base pair sequence containing core-type Int binding sites and arm-type Int binding sites as well as sites for auxiliary proteins integration host factor (IHF), FIS and excisionase (Xis). See Landy, Curr. Opin. Biotech. 3:699 707 (1993). Further examples of recognition sequences include loxP site mutants, variants or derivatives such as loxP511 (see U.S. Pat. No. 5,851,808); dif sites; dif site mutants, variants or derivatives; psi sites; psi site mutants, variants or derivatives; cer sites; and cer site mutants, variants or derivatives. See also, for example, US20100267128 and WO 01/11058, incorporated by reference herein in their entirety. Other systems providing recombination sites and recombination proteins for use in the invention include the FLP/FRT system from Saccharomyces cerevisiae, the resolvase family (e.g., RuvC, yi, TndX, TnpX, Tn3 resolvase, Hin, Hjc, Gin, SpCCE1, ParA, and Cin), and IS231 and other Bacillus thuringiensis transposable elements. Other suitable recombination systems for use in the present invention include the XerC and XerD recombinases and the psi, dif and cer recombination sites in Escherchia coli. Other suitable recombination sites may be found in U.S. Pat. No. 5,851,808, which is specifically incorporated herein by reference.

This embodiment can be used for recombinational cloning, for example using the Gateway® Cloning System described in published U.S. Pat. Application No. US20100267128, and in U.S. application Ser. No. 09/177,387, filed Oct. 23, 1998; U.S. application Ser. No. 09/517,466, filed Mar. 2, 2000; and U.S. Pat. Nos. 5,888,732 and 6,143,557, all of which are specifically incorporated herein by reference. In brief, the Gateway® Cloning System utilizes vectors that contain at least one recombination site to clone desired nucleic acid molecules in vivo or in vitro. In one embodiment, the system utilizes vectors that contain at least two different site-specific recombination sites based on the bacteriophage lambda system (e.g., att1 and att2) that are mutated from the wild-type (att0) sites. Each mutated site has a unique specificity for its cognate partner att site (i.e., its binding partner recombination site) of the same type (for example attB1 with attP 1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the Gateway® system by replacing a selectable marker (for example, Tse2) flanked by att sites on the recipient plasmid molecule, sometimes termed the Destination Vector. Desired clones are then selected by transformation of a Tse2 sensitive host strain and positive selection for a marker on the recipient molecule. Tse2 is toxic to both prokaryotic and eukaryotic cells, and thus Tse2 sensitive host strains include both prokaryotic and eukaryotic cells.

In one embodiment, the vector contains a Tse2 gene flanked by one or more restriction enzyme sites or recombination sites. Recombination sites include, but are not limited to, attB, attP, attL, and attR. This vector is designed such that the DNA fragment of interest (such as, for example, a PCR product) will replace the Tse2 located between the two flanking sites. If the DNA fragment of interest is present in the vector, the cells containing the vector survive, as the Tse2 gene will no longer be present on the desired recombinant vector. If the gene of interest is not present, the Tse2 gene will prevent survival of the cell carrying the undesired vector. Thus, only cells containing positive clones with the DNA fragment of interest will be viable, and easily selected for.

In one embodiment, the vector comprises at least one inactive fragment of the Tse2 gene, wherein a functional Tse2 gene is rescued when the inactive fragment is recombined across at least one recombination site with a second DNA segment comprising another inactive fragment of the Tse2 gene.

In another embodiment, the vector contains a dual selection cassette, wherein the vector comprises a first gene encoding Tse2, and a second gene encoding a second selectable marker, such as an antibiotic resistance gene or a second “death” gene encoding a second toxic protein. The antibiotic resistance gene can be selected from either bacterial or eukaryotic genes, and can promote resistance to ampicillin, kanamycin, tetracycline, cloramphenicol, and others known in the art. The second death gene can be any suitable death gene, including but not limited to, rpsL, tetAR, pheS, thyA, lacY, gata-1, ccdB, and sacB. The second death gene can also be selected from either prokaryotic or eukaryotic toxic genes. This dual selection cassette is flanked by at least one restriction site or recombination site, such that the DNA fragment of interest will replace the dual selection cassette located between the two sites in the desired recombination or ligation event. If the DNA fragment of interest is present, the cells containing the vector survive, as the Tse2 gene will no longer be present on the desired recombinant vector. If the gene of interest is not present, the vector will still contain the Tse2 gene and will prevent survival of the cell carrying the undesired vector. This dual selection cassette can thus be used for any double negative selection strategy as desired by one of ordinary skill in the art. In one embodiment, the Tse2 gene double negative selection strategy is used when use of multiple antibiotics is not be compatible with the particular selection design.

As a non-limiting example, the vector contains a dual selection cassette comprising the Tse2 gene as well as a cloramphenicol resistance gene under control of at least one promoter. The vector is cut using restriction enzymes both upstream and downstream of the dual selection cassette. Optionally, the linearized vector can be gel purified to remove the excised dual selection cassette DNA from the reaction. DNA containing the DNA fragment of interest and appropriate restriction enzyme sites, such as a PCR product, is then combined with the linearized vector in a ligation reaction. Positive clones will be chloramphenicol sensitive and viable (Tse2 negative), due to the replacement of the dual selection cassette with the DNA fragment of interest.

In another embodiment, the vector contains at least one recombination site within the Tse2 gene or corresponding regulatory element (e.g. promoter or enhancer), such that a desired recombination event will disrupt the expression of the Tse2 gene from the vector. The location of the recombination site should be chosen such that if the desired recombination event occurs, the resulting Tse2 gene will be inactive and the cell containing the desired vector will survive. If the desired recombination event does not occur, the Tse2 gene will remain intact and the cell containing the undesired vector will not survive.

In another embodiment, the vector contains at least one recombination site within the Tse2 gene or corresponding regulatory element (e.g. promoter or enhancer), such that an undesired recombination event will produce an intact and functional Tse2 gene, which will result in the death of the cell containing the undesired vector.

In another embodiment, the Tse2 gene is fragmented on multiple vectors, with shared restriction enzyme sequences or recombination site sequences connecting the gene fragments. The vectors are designed and arranged such that an undesired recombination event or ligation event will result in the creation of an intact Tse2 gene on the undesired plasmid, thus resulting in the death of the cells containing the undesired vector with the functional Tse2 gene.

In another embodiment, the vectors are ones suitable for topoisomerase-mediated cloning, as described in U.S. Pat. Nos. 5,766,891 and 7,550,295, and/or TA cloning, as disclosed in U.S. Pat. No. 5,827,657, both references incorporated by reference herein in their entirety. In certain embodiments, the vectors suitable for topoisomerase or TA-mediated cloning are linearized, such that the vectors are optimized for most efficient integration of the DNA fragment of interest. These preparations are described in the referenced patents.

Briefly, topoisomerase-mediated cloning relies on the principle that Taq polymerase has a non-template-dependent terminal transferase activity that adds a single deoxyadenosine (A) to the 3′ ends of PCR products. For example, topoisomerase I from Vaccinia virus binds to duplex DNA at specific sites (CCCTT) and cleaves the phosphodiester backbone in one strand. The energy from the broken phosphodiester backbone is conserved by formation of a covalent bond between the 3′ phosphate of the cleaved strand and a tyrosyl residue (Tyr-274) of topoisomerase I. The phospho-tyrosyl bond between the DNA and enzyme can subsequently be attacked by the 5′ hydroxyl of the original cleaved strand, reversing the reaction and releasing topoisomerase. In one embodiment, the vectors of the invention comprise a linear vector containing single, overhanging 3′ deoxythymidine (T) residues, with a topoisomerase I covalently bound to the vector (referred to as “activated vector”). This allows PCR inserts to ligate efficiently with the vector.

In another embodiment, the vectors are designed for topoisomerase or TA cloning, such that the topoisomerase or TA cleavage sites are located within the Tse2 gene. In this embodiment, the vector can be used for negative selection of clones that are lacking a desired DNA insert. After conducting the topoisomerase or TA reaction, the vectors that contain a desired DNA insert will have a disrupted and inactive Tse2 gene, thus allowing the cells containing that vector to survive. However, if the vector circularizes at the cleavage sites without incorporating an insert, the Tse2 gene will be reformed and active, thus producing the toxic Tse2 protein and killing the cell. In further embodiments, the topoisomerase or TA site will be flanked with restriction enzyme sites and/or sequencing primer sites.

In another embodiment, the TA or TOPO cloning strategies can be combined, as disclosed, for example, in U.S. Pat. No. 6,916,632, incorporated herein for reference in its entirety.

In another aspect of the invention that can be combined with any other embodiment herein, the recombinant vector may comprise a gene encoding a Tse2 antidote operatively linked to a regulatory sequence. The antidote can be any expression product capable of interfering with the cytotoxic activity of Tse2, including but not limited to Tse2 antisense constructs, Tse2-binding aptamers, and Tse2-binding polypeptides. Such vectors can be used, for example, as markers in a cell whose survivability can be conditionally controlled by controlling conditions under which the antidote polypeptide is expressed. In a preferred embodiment that can be combined with any other embodiment herein, the second gene codes for type VI secretion immunity protein 2 (Tsi2), disclosed in the examples that follow as an antidote to Tse2. In one preferred embodiment, the second gene comprises or consists of a nucleotide sequence that can encode a P. aeruginosa Tsi2 amino acid sequence according to SEQ ID NO:10. In another preferred embodiment, the second gene comprises or consists of a nucleotide sequence according to SEQ ID NO:9.

Closely related Tsi2 genes and Tsi2 proteins are present in other P. aeruginosa strains, with variable positions noted in SEQ ID NO:11. Thus, in another preferred embodiment, the second gene comprises or consists of a nucleotide sequence that can encode an amino acid sequence according to SEQ ID NO:11. In another preferred embodiment, the second gene comprises or consists of a nucleotide sequence according to SEQ ID NO:12.

As used herein, “Tsi2” includes functional equivalents (truncations, mutants, etc.) thereof, wherein such equivalents maintain their ability to confer immunity upon cells expressing Tse2, as described herein. Methods for identifying such functional equivalents are disclosed herein and a variety of such functional equivalents are disclosed. For example, the inventors have discovered that residues 60-77 of Tsi2 can be removed while retaining its Tse2 immunity activity. Thus, in a further embodiment, the second gene comprises or consists of a nucleotide sequence that can encode an amino acid sequence according to SEQ ID NO:13.

The inventors have further identified a series of Tsi2 mutant polypeptides that retain Tse2 immunity function. Specifically, the inventors have shown that the Tsi2 mutants having single mutations described below retain Tse2 immunity activity, showing that Tsi2 is resilient and its interactions with Tse2 are robust. Thus, in another embodiment, the second gene comprises or consists of a nucleotide sequence that can encode an amino acid sequence according to SEQ ID NO:10, 11, or 13 with 1, 2, 3, 4, 5, or more amino acid substitutions. Exemplary positions at which such substitutions can be made (referring to SEQ ID NO:10-12 numbering) are amino acid residues 2, 4, 6, 7, 8, 10, 11, 13, 14, 18, 20, 21, 25, 27, 28, 29, 30, 32, 33, 36, 38, 39, 42, 44, 45, 46, 47, 49, 50, 52, 56, 57, 59, and 61.

As a non-limiting example, the Tsi2 gene is described as an exemplary Tse2 antidote in the embodiments herein. However, this should not be read a limiting the invention in any way. Any Tse2 antidote could be substituted for the disclosed embodiments, including but not limited to Tse2 antisense constructs, Tse2-binding aptamers, and Tse2-binding polypeptides.

The Tsi2 gene can be under the regulatory control of any promoter desired, including but not limited to those disclosed above for Tse2, such as the various inducible promoters disclosed above, as well as baculovirus polyhedrin, SP6, metallothionein I, Autographa californica nuclear polyhidrosis virus, Semliki Forest virus, Tet, CMV, Gall, Ga110, and T7 promoters.

In one embodiment, the Tsi2 gene is included on a vector which will, when expressed, confer immunity to a cell which is expressing Tse2. In a cell line which is expressing Tse2 in the absence of Tsi2, the cells will not survive. Also provided herein is the Tsi2 gene under the control of an inducible promoter, as described above. If a Tse2-expressing cell receives the vector which expresses the Tsi2 gene, that prokaryotic or eukaryotic cell will survive, while such cells that do not express the Tsi2 gene will not survive. As noted in the examples herein, without intending to be bound to any particular mechanism, the mechanism of Tsi2 inhibition of Tse2 is likely to involve physical association of the proteins.

In another embodiment, the Tsi2 gene can be used as a marker for a desired recombination or ligation event. In a non-limiting example, the vector contains a Tsi2 gene flanked by one or more recombination sites. The DNA fragment of interest is inserted into a site on the vector, such that the fragment does not disrupt the Tsi2 gene but is contained within the recombination sites. In another embodiment, a topoisomerase or TA site is included within the flanking sites, but outside the Tsi2 gene, to facilitate DNA fragment insertion. The vector containing the DNA fragment of interest is then combined with a second vector containing matching recombination sites, such that a positive recombination event will move the DNA fragment of interest and the Tsi2 gene into the new vector, which can then be selected for survival in cells expressing Tse2. In another non-limiting example, the vector contains a Tsi2 gene flanked by one or more restriction sites. The DNA fragment of interest is inserted into a site on the vector, such that the fragment does not disrupt the Tsi2 gene but is contained within the restriction sites. The vector containing the DNA fragment of interest and a second cloning vector are then digested with one or more restriction enzymes, followed by a ligation reaction. A positive ligation event will move the DNA fragment of interest and the Tsi2 gene into the second cloning vector, which can then be selected for survival in cells expressing Tse2. In another embodiment, different antibiotic resistance genes can also be used on the plasmids such that double selection can be employed by one of ordinary skill in the art.

In one embodiment, the vector comprises a Tsi2 gene in an inactive form, such as a truncated form. This vector can be used, for example, in methods for rescuing the activity of the Tsi2 gene such that vectors which contain a functional Tsi2 gene also contain the DNA fragment of interest (as described herein). The functional Tsi2 can be rescued by recombination, integration, or other events or reactions as described herein. Vectors can be readily designed for the particular experiment by one of ordinary skill in the art.

In another aspect of the invention, the invention provides herein a recombinant vector which contains a truncated or inactive version of the antitoxin (Tsi2) gene is present on the vector. In a non-limiting example, the vector may be in linear form. In order to restore the function of the Tsi2 gene, a short sequence of nucleotides are added to the end of the DNA fragment of interest to be cloned. This sequence corresponds to the truncated sequence of the Tsi2 gene, such that this sequence attached to the DNA fragment of interest will bind with the truncated Tsi2 gene, thus restoring an active antitoxin protein able to counteract the action of the Tse2 protein. The short sequence is incorporated to the DNA fragment using one modified PCR primer. This system allows for the positive selection of recombinant plasmids only and for the selection of the correct orientation of the cloned fragment in the vector, as only one of the two possible orientations will restore an active Tsi2 gene.

In another embodiment, the truncation of the Tsi2 gene is located within the regions as defined in the invention as required for Tsi2 antidote function. For example, as described herein, the inventors have discovered that residues 60-77 of Tsi2 can be removed while retaining its Tse2 immunity activity. As such, the truncation of Tsi2 must be outside those residues in order to produce an inactive Tsi2 protein.

In another embodiment, the vector containing the truncated, inactive Tsi2 gene is circular.

In another embodiment, the invention provides a recombinant vector, in which a gene encoding Tsi2 would be functional only after proper elimination of an antibiotic resistance gene or additional cell death gene. Any antibiotic resistance gene or additional death gene could be used in this embodiment. In one non-limiting example, the Tsi2 locus is split into two parts on the same plasmid containing a common sequence, and cloned in the 5′ and 3′ regions flanking the kanamycin resistance gene. After digestion at a restriction site located inside the kanamycin resistance gene and transformation of Tse2 expressing cells with linear DNA, a fully functional Tsi2 would assemble through homologous recombination. Only bacteria or eukaryotic cells containing a recombinant plasmid with a functional Tsi2 can grow upon transformation. For a description of this strategy using the ccdB gene, see Peubez, et al. Microbial Cell Factories 2010, 9:65, which is incorporated by reference.

In another embodiment, the Tsi2 locus is split into two or more parts on two or more plasmids.

In another embodiment, the Tsi2 locus is split into two or more parts on two or more plasmids or integrated into the chromosome of a cell.

In another embodiment, the vector comprises one or more unique restriction enzyme recognition sites, wherein cloning of a nucleic acid insert into the one or more unique restriction enzyme recognition sites disrupts expression of the Tsi2 antidote gene. The vectors of this embodiment can be used as cloning vehicles, since cloning of an insert into the one or more restriction sites in the vector interrupts Tsi2 antidote gene expression and provide an easily selectable marker. Cells with vectors containing no insert survive, those with insert die.

In another embodiment, the invention comprises a first vector that contains the Tse2 gene according to any embodiment disclosed herein, and a second vector that contains the Tsi2 gene according to any embodiment disclosed herein.

In another embodiment, the invention comprises a vector that contains the Tse2 gene according to any embodiment disclosed herein, and contains the Tsi2 gene according to any embodiment disclosed herein.

In one embodiment, the vector contains a Tsi2 gene such that loss of the expression of the Tsi2 gene renders the cell non-viable.

In one embodiment, the invention provides one or more vectors containing Tse2 and Tsi2 for use in the Gateway recombination system as described herein. The vector is designed such that if the desired recombination event does not occur, the Tse2 will be active on the vector, while Tsi2 will be inactive, and the cells containing the vector will die. If the desired recombination event does occur, the vector will carry both the Tse2 and Tsi2 genes, conferring the Tse2 antidote to the cell containing the vector, and the cell will survive. In one embodiment, one vector can comprise both Tse2 and Tsi2 genes. In another embodiment, each gene can be found on a separate vector. This strategy can be used to replace one or more antibiotic resistance genes in the Gateway system.

In one embodiment, the vector contains the Tsi2 antidote gene. The vector is transformed into cells that contain a stably integrated Tse2 gene, but which is controlled by an inactive promoter. For example, the Tse2 gene is controlled by a T7 promoter, but integrated into bacteria that are lacking the T7 RNA polymerase gene. Design and synthesis of nucleic acid sequences and preparation of vectors comprising such sequences is well within the level of skill in the art.

In another embodiment, one vector contains the Tsi2 gene and one vector contains the Tse2 gene. Both of these vectors can be found episomally in a single cell.

In addition to components of the vector which may be required for expression of Tse2 (and Tse2 antidote, if present), vectors may also include any other suitable control elements, including but not limited to origin of replication, primer sites, e.g., for PCR, transcriptional and/or translational initiation and/or regulation sites, recombinational signals, replicons, other selection markers, antibiotic resistance genes, etc. In one embodiment, the replication sequence renders the vector capable of episomal and chromosomal replication, such that the vector is capable of self-replication as an extrachromosomal unit and of integration into the chromosome, either due to the presence of a translocatable sequence, such as an insertion sequence or transposon, due to substantial homology with a sequence present in the chromosome or due to non-homologous recombinational events. The replication sequence or replicon will be one recognized by the transformed host and is derived from any convenient source, such as from a plasmid, virus, the host cell, e.g., an autonomous replicating segment, by itself, or in conjunction with a centromere, or the like. The particular replication sequence is not critical to the subject invention and various sequences may be employed. Conveniently, a replication sequence of a virus can be employed.

In all embodiments, each individual nucleic acid segment may comprise a variety of sequences including, but not limited to sequences suitable for use as primer sites (e.g., sequences for which a primer such as a sequencing primer or amplification primer may hybridize to initiate nucleic acid synthesis, amplification or sequencing), transcription or translation signals or regulatory sequences such as promoters and/or enhancers, ribosomal binding sites, Kozak sequences, start codons, termination signals such as stop codons, origins of replication, recombination sites (or portions thereof), selectable markers, and genes or portions of genes to create protein fusions (e.g., N-terminal or C-terminal) such as GST, GUS, GFP, YFP, CFP, maltose binding protein, 6 histidines (HIS6), epitopes, haptens and the like and combinations thereof. The vectors used for cloning such segments may also comprise these functional sequences (e.g., promoters, primer sites, etc.). After combination of the segments comprising such sequences and optimally the cloning of the sequences into one or more vectors, the molecules may be manipulated in a variety of ways, including sequencing or amplification of the target nucleic acid molecule (i.e., by using at least one of the primer sites introduced by the integration sequence), mutation of the target nucleic acid molecule (i.e., by insertion, deletion or substitution in or on the target nucleic acid molecule), insertion into another molecule by homologous recombination, transcription of the target nucleic acid molecule, and protein expression from the target nucleic acid molecule or portions thereof (i.e., by expression of translation and/or transcription signals contained by the segments and/or vectors). Cloning vectors can be stored in a freezer, refrigerator, liquid nitrogen, or any other methods known to one of ordinary skill in the art.

In another aspect, the present invention provides recombinant host cells comprising the recombinant vector of any embodiment or combination of embodiments of any aspect of the invention. A “host,” as the term is used herein, can be any prokaryotic or eukaryotic organism that can be genetically engineered to express heterologous Tse2 (and Tse2 antidote, if present) including but not limited to bacterial (such as E. coli), algal, fungal (such as yeast), insect, invertebrate, plant, and mammalian cell types. For examples of such hosts, see Maniatis et al., Molecular Cloning: A Laboratory Manual, Cold Spring Harbor Laboratory, Cold Spring Harbor, N.Y. (1982). The host cells of this aspect of the invention can be used, for example, in the methods of the invention discussed herein.

In one embodiment, the bacteria or eukaryotic cell contains the Tse2 gene or gene fragment stably integrated in the chromosome under the control of a selected promoter.

In another embodiment, the bacteria or eukaryotic cell contains the Tse2 gene or gene fragment carried in a vector under the control of a selected promoter.

In one embodiment, the bacteria or eukaryotic cell contains the Tsi2 gene or gene fragment stably integrated in the chromosome under the control of a selected promoter.

In another embodiment, the bacteria or eukaryotic cell contains the Tsi2 gene or gene fragment carried in a vector under the control of a selected promoter.

In another aspect, the present invention provides methods for selectable cloning, comprising culturing the recombinant host cell of any embodiment of another aspect of the invention under conditions suitable for expression of Tse2 from the recombinant vector if no insert is present, and selecting those cells that grow as comprising recombinant vectors with the insert cloned into the expression vector. In one embodiment, the vector comprises one or more unique restriction enzyme recognition sites, and wherein cloning of a nucleic acid insert into the one or more unique restriction enzyme recognition sites disrupts expression of the first gene, and cloning of an insert into the one or more restriction sites in the vector interrupts Tse2 expression and provide an easily selectable marker—cells transfected with vectors containing no insert have their growth inhibited by Tse2 expression (so long as they do not endogenously express an antidote to Tse2), and those with inserts do not. In another embodiment, the recombinant vector comprises one or more recombination sites flanking the Tse2 gene.

In one embodiment, the recombinant vector comprises at least a first and a second recombination site flanking a first gene coding for Tse2 operatively linked to a regulatory sequence, wherein said first and second recombination sites do not recombine with each other. In this embodiment, nucleic acid fragments to be cloned are flanked by recombination sites and cloned/subcloned by replacing the Tse2 selectable marker flanked by recombination sites on the recombinant vector. Desired clones are then selected by transformation of a Tse2 sensitive host strain and any positive selection for a marker on the recipient molecule. Tse2 is toxic to both prokaryotic and eukaryotic cells, and thus Tse2 sensitive host strains include both prokaryotic and eukaryotic cells. Since Tse2 is toxic in both prokaryotic and eukaryotic cells, such selectable cloning can be carried out in any prokaryotic or eukaryotic host cell, including but not limited to bacterial (such as E. coli), algal, fungal (such as yeast), insect, invertebrate, plant, and mammalian cell types. Conditions for cell culture suitable for Tse2 expression can be determined by those of skill in the art based on a variety of factors, including the specific host cell, regulatory sequence(s), and vector design in light of the teachings herein.

In another aspect, the present invention provides methods for producing a cloning vector that lacks an insert, comprising culturing the recombinant host cell of any embodiment of the second aspect of the invention under conditions suitable for vector replication and expression of Tse2, wherein the recombinant host cells further express a Tse2 antidote, and isolating vector from the host cells. These methods permit large scale production of the vectors of any embodiment of the present invention. The antidote can be any expression product capable of interfering with the cytotoxic activity of Tse2, including but not limited to Tse2 antisense constructs, Tse2-binding aptamers, and Tse2-binding polypeptides. In a preferred embodiment that can be combined with any other embodiment herein, the Tse2 antidote comprises any embodiment of Tsi2 disclosed herein. Conditions for cell culture suitable for vector replication can be determined by those of skill in the art based on a variety of factors, including the specific host cell, regulatory sequence(s), and vector design in light of the teachings herein.

In another aspect, the present invention provides host cells comprising in their genome, a first recombinant gene coding for Tse2 operatively linked to a regulatory sequence. In one non-limiting embodiment, the recombinant cell comprises a second gene encoding an antidote (such as Tsi2) on a plasmid or a mobile genetic element, and selection for its antidote properties (i.e.: Tse2 immunity) maintain that element. In another embodiment, the recombinant host cell comprises a first gene encoding functional Tse2 on a plasmid, wherein the recombinant host cell comprises the second gene expressing Tsi2 to permit Tse2 plasmid propagation in the host cell. In this embodiment, the second gene can be present on the same or different plasmid, another extra-chromosomal element, or chromosomally integrated. The first gene and second gene are “recombinant” in that the host cell does not endogenously express Tse2 or a Tse antidote, and thus Tse2 expression requires recombinant expression of Tse2, and antidote expression requires recombinant expression of the antidote.

As used herein, “in its genome” includes chromosomal insertion and extra-chromosomal elements, such as plasmids or viral vectors. Thus, in one preferred embodiment, the first recombinant gene and/or the second recombinant gene are present extra-chromosomally. In a further preferred embodiment, the first recombinant gene and/or the second recombinant gene are present as chromosomal insertions. In embodiments in which the second gene coding for an antidote for Tse2 is present, the second gene may be on the same, or alternatively, on a different extra-chromosomal element than the first gene, or, alternatively, linked or unlinked to the first gene in the genome. In other embodiments, one of the first and second genes can be a chromosomal insertion, while the other of the first and second genes can be an extra-chromosomal element. In embodiments having the first and second genes are on the same plasmid, the genes can be closely linked. In another embodiment, the first and second genes are on the same plasmid and are not closely linked.

In embodiments where the host cells do not comprise a second recombinant gene coding for an antidote for Tse2, the Tse2 regulatory sequences are preferably controllable, to control Tse2 expression. In exemplary embodiments where the host cells do comprise a second recombinant gene coding for an antidote for Tse2, the Tse2 regulatory sequence may be inducible and/or the antidote regulatory sequence may be constitutive, to control Tse2 expression.

In another non-limiting embodiment, the recombinant cell (such as a mammalian cell) comprises a first gene encoding Tse2 on one plasmid, and a second gene encoding Tsi2 on a second plasmid. In this embodiment, Tsi2 can be used as a selectable marker on an expression vector, wherein the Tse2-expressing host cell is introduced into the cells, and only cells expressing Tsi2 will be able to grow. In one embodiment, the regulatory region for Tse2 is inducible, and that growth of the cells post-introduction occurs under inducing condition. In this embodiment, the second vector may further comprise a recombinant nucleic acid of interest for expression or other purposes. In embodiments where the Tse2 regulatory element is controllable, control of Tse2 expression can be used to maintain the Tsi2 plasmid and/or to select for its integration, providing a way to make stable cells without using an antibiotic.

In another aspect, the vectors can be provided in a kit. The present invention also relates to kits for carrying out the methods of the invention, and particularly for use in creating the product nucleic acid molecules of the invention or other linked molecules and/or compounds of the invention (e.g., protein-protein, nucleic acid-protein, etc.), or supports comprising such product nucleic acid molecules or linked molecules and/or compounds. The invention also relates to kits for adding and/or removing and/or replacing nucleic acids, proteins and/or other molecules and/or compounds, for creating and using combinatorial libraries of the invention, and for carrying out homologous recombination (particularly gene targeting) according to the methods of the invention.

The kits of the invention may also comprise further components for further manipulating the recombination site-containing molecules and/or compounds produced by the methods of the invention. The kits of the invention may comprise one or more nucleic acid molecules of the invention (particularly starting molecules comprising one or more recombination sites and optionally comprising one or more reactive functional moieties), one or more molecules and/or compounds of the invention, one or more supports of the invention and/or one or more vectors of the invention. Such kits may optionally comprise one or more additional components selected from the group consisting of one or more host cells (e.g., two, three, four, five etc.), one or more reagents for introducing (e.g., by transfection or transformation) molecules or compounds into one or more host cells, one or more nucleotides, one or more polymerases and/or reverse transcriptases (e.g., two, three, four, five, etc.), one or more suitable buffers (e.g., two, three, four, five, etc.), one or more primers (e.g., two, three, four, five, seven, ten, twelve, fifteen, twenty, thirty, fifty, etc.), one or more terminating agents (e.g., two, three, four, five, seven, ten, etc.), one or more populations of molecules for creating combinatorial libraries (e.g., two, three, four, five, seven, ten, twelve, fifteen, twenty, thirty, fifty, etc.) and one or more combinatorial libraries (e.g., two, three, four, five, seven, ten, twelve, fifteen, twenty, thirty, fifty, etc.). The kits of the invention may also contain directions or protocols for carrying out the methods of the invention.

In another aspect the invention provides kits for joining, deleting, or replacing nucleic acid segments, these kits comprising at least one component selected from the group consisting of (1) one or more recombination proteins or compositions comprising one or more recombination proteins, and (2) at least one nucleic acid molecule comprising one or more recombination sites (preferably a vector having at least two different recombination specificities). The kits of the invention may also comprise one or more components selected from the group consisting of (a) additional nucleic acid molecules comprising additional recombination sites; (b) one or more enzymes having ligase activity; (c) one or more enzymes having polymerase activity; (d) one or more enzymes having reverse transcriptase activity; (e) one or more enzymes having restriction endonuclease activity; (f) one or more primers; (g) one or more nucleic acid libraries; (h) one or more supports; (i) one or more buffers; (j) one or more detergents or solutions containing detergents; (k) one or more nucleotides; (l) one or more terminating agents; (m) one or more transfection reagents; (n) one or more host cells; and (o) instructions for using the kit components.

In one embodiment, kits of the invention contain compositions comprising at least one linearized or circular vector containing the Tse2 or Tsi2 gene. In some embodiments, the linearized vector contained in the kit is treated such that the ends of the vector are resistant to binding to the other ends of the vector.

In other embodiments, the present invention relates to a kit comprising a carrier or receptacle being compartmentalized to receive and hold therein at least one container, wherein a first container contains linear or circular DNA molecule comprising a vector having at least one DNA fragment of the Tse2 gene sequence, as described herein. In another embodiment, the vector contained in the kit has at least one DNA fragment of the Tsi2 gene sequence, as described herein. In another embodiment, the kit contains both vectors which have at least one DNA fragment of the Tse2 sequence and vectors that have at least one DNA fragment of the Tsi2 sequence.

All embodiments and combinations of embodiments of Tse2 and Tsi2 disclosed above can be used in this aspect of the invention.

### EXAMPLE 1

### Summary

The functional spectrum of a secretion system is defined by its substrates. Here we analyzed the secretomes of Pseudomonas aeruginosa mutants altered in regulation of the Hcp Secretion Island-1-encoded type VI secretion system (H1-T6SS). We identified three substrates of this system, proteins Tse1-3 (type six exported 1-3), which are co-regulated with the secretory apparatus and secreted under tight posttranslational control. The Tse2 protein was found to be the toxin component of a toxin-immunity system, and to arrest the growth of prokaryotic and eukaryotic cells when expressed intracellularly. In contrast, secreted Tse2 had no effect on eukaryotic cells; however, it provided a major growth advantage for P. aeruginosa strains, relative to those lacking immunity, in a manner dependent on cell contact and the H1-T6SS. This demonstration that the T6SS targets a toxin to bacteria helps reconcile the structural and evolutionary relationship between the T6SS and the bacteriophage tail and spike.

### Introduction

Secreted proteins allow bacteria to intimately interface with their surroundings and other bacteria. The importance and diversity of secreted proteins is reflected in the multitude of pathways bacteria have evolved to enable their export (Abdallah et al., 2007; Filloux, 2009). Large multi-component secretion systems, including types III and IV secretion, have been the focus of a great deal of study because in many organisms they are specialized for effector export and they have the remarkable ability to directly translocate proteins from bacterial to host cell cytoplasm via a needle-like apparatus (Cambronne and Roy, 2006). The recently described type VI secretion system (T6SS) is another specialized system, however its physiological role and general mechanism remain poorly understood (Bingle et al., 2008).

Studies of T6SSs indicate that a functional apparatus requires the products of approximately 15 conserved and closely linked genes, and is strongly correlated to the export of a hexameric ring-shaped protein belonging to the hemolysin co-regulated protein (Hcp) family (Filloux, 2009; Mougous et al., 2006). Hcp proteins are required for assembly of the secretion apparatus and they interact with valine-glycine repeat (Vgr) family proteins, which are also exported by the T6SS. The function of the Hcp/Vgr complex remains unclear, however it is believed that the proteins are extracellular structural components of the secretion apparatus. Recent X-ray crystallographic insights into Hcp and Vgr-family proteins show that they are similar to bacteriophage tube and tail spike proteins, respectively (Leiman et al., 2009; Pell et al., 2009). These findings prompted speculation that the T6SS is evolutionarily, structurally, and mechanistically related to bacteriophage. According to this model, the T6SS assembles as an inverted phage tail on the surface of the bacterium, with the Hcp/Vgr complex forming the distal end of the cell-puncturing device. Another notable conserved T6S gene product is ClpV, a AAA+-family ATPase that has been postulated to provide the energy necessary to drive the secretory apparatus (Mougous et al., 2006). The roles of the remaining conserved T6S proteins remain largely unknown.

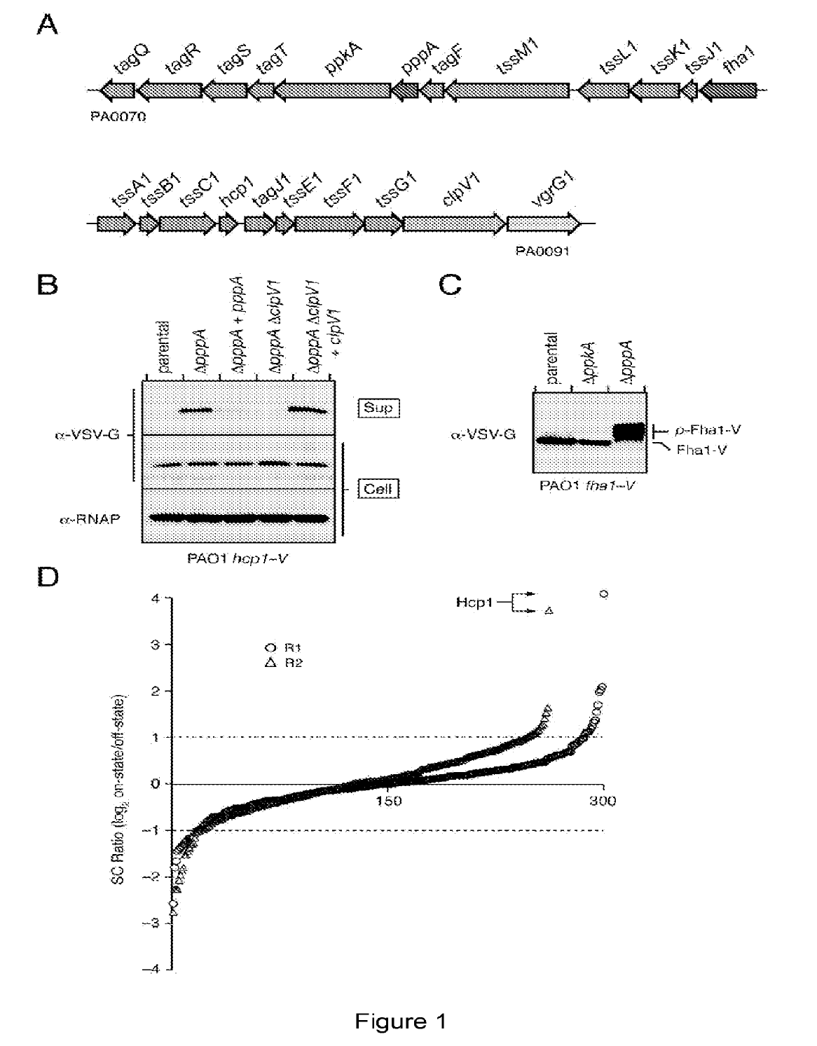

Nonconserved genes encoding predicted accessory elements are also linked to most T6SSs (Bingle et al., 2008). In the HSI-I-encoded T6SS of Pseudomonas aeruginosa (H1-T6SS) (FIG. 1A), these genes encode elements of a posttranslational regulatory pathway that strictly modulates the activity of the secretion system through changes in the phosphorylation state of a forkhead-associated domain protein, Fha1 (Mougous et al., 2007). Phosphorylation of Fha1 by a transmembrane serine-threonine Hanks-type kinase, PpkA, triggers Hcp1 secretion. PppA, a PP2C-type phosphatase, antagonizes Fha1 phosphorylation.

The T6SS has been linked to a myriad of processes, including biofilm formation (Aschtgen et al., 2008; Enos-Berlage et al., 2005), conjugation (Das et al., 2002), quorum sensing regulation (Weber et al., 2009), and both promoting and limiting virulence (Filloux, 2009). The P. aeruginosa H1-T6SS has been implicated in the fitness of the bacterium in a chronic infection; mutants in conserved genes in this secretion system failed to efficiently replicate in a rat lung chronic infection model and the system was shown to be active in cystic fibrosis (CF) patient infections (Mougous et al., 2006; Potvin et al., 2003). The H1-T6SS is also co-regulated with other chronic infection virulence factors such as the psl and pel loci, which are involved in biofilm formation (Goodman et al., 2004; Ryder et al., 2007).

How the apparently conserved T6SS architecture can participate in such a wide range of activities is not clear. At least one mechanism by which the secretion system can exert its effects on a host cell has been garnered from studies of Vibrio cholerae. A T6S-associated VgrG-family protein of this organism contains a domain with actin-crosslinking activity that is translocated into host cell cytoplasm in a process requiring endocytosis and cell-cell contact (Ma et al., 2009; Pukatzki et al., 2007; Satchell, 2009). The subset of VgrG-family proteins that contain non-structural domains with conceivable roles in pathogenesis have been termed “evolved” VgrG proteins (Pukatzki et al., 2007). This configuration, wherein an effector domain is presumably translocated into host cell cytoplasm by virtue of its fusion to the T6S cell puncturing apparatus, is intriguing, but it is likely not general; a multitude of organisms containing T6SSs do not encode “evolved” VgrG proteins (Boyer et al., 2009; Pukatzki et al., 2009).

Key to understanding the function of the T6SS—as with any secretion system—is to identify and characterize the protein substrates that it exports. EvpP from Edwardsiella tarda and RbsB from Rhizobium leguminosarum are proposed substrates of the system; however, inconsistent with anticipated properties of T6S substrates, RbsB contains an N-terminal Sec secretion signal, and EvpP stably associates with a component of the secretion apparatus (Bladergroen et al., 2003; Pukatzki et al., 2009; Zheng and Leung, 2007).

In this study, we identified three proteins, termed Tse1-3 (type VI secretion exported 1-3), that are substrates of the H1-T6SS of P. aeruginosa. We showed that one of these, Tse2, is the toxin component of a toxin-immunity system, and that it is able to arrest the growth of a variety of prokaryotic and eukaryotic organisms. Despite the promiscuity of toxin expressed intracellularly, we found that H1-T6SS-exported Tse2 was specifically targeted to bacteria. In growth competition experiments, immunity to Tse2 provided a marked growth advantage in a manner dependent on intimate cell-cell contact and a functional H1-T6SS. The ability of the secretion system to efficiently target Tse2 to a bacterium, and not to a eukaryotic cell, suggests that T6S may play a role in the delivery of toxin and effector molecules between bacteria.

### Results

### Design and Characterization of H1-T6SS On- and Off-State Strains

Under laboratory culturing conditions, activation of the H1-T6SS is strongly repressed at the posttranslational level by the phosphatase PppA (FIG. 1A). We have shown that inactivation of pppA leads to Hcp1 export, and that this could reflect triggering of the “on-state” in the secretory apparatus (Hsu et al., 2009; Mougous et al., 2007). These observations led us to predict that additional components of the apparatus, and even substrates of the secretion system, are also exported in this state. To identify these proteins, we sought to compare the secretomes of ΔpppA and ΔclpV1. The latter lacks the H1-T6SS ATPase, ClpV1, and therefore remains in the “off-state” (FIG. 1A) (Mougous et al., 2006).

To probe whether the on-state and off-state mutations could modulate the activity of the H1-T6SS, we assayed their effect on Hcp1 secretion in P. aeruginosa PAO1 hcp1-V (where present, -V denotes a fusion of the indicated gene to a sequence encoding the vesicular stomatitis virus G epitope). As expected, the deletion of pppA promoted Hcp1 secretion and Fha1 phosphorylation relative to the parental strain (FIGS. 1B and C). Since the wild-type strain does not secrete Hcp1 to detectable levels, the effects of ΔclpV1 were gauged using the ΔpppA background. Introduction of the clpV1 deletion to ΔpppA abrogated Hcp1 secretion and this effect was fully complemented by ectopic expression of clpV1 (FIG. 1B). These data indicate that pppA and clpV1 deletions are sufficient to activate and inactivate the H1-T6SS secretion system, respectively.

### Mass Spectrometric Analysis of On- and Off-State Secretomes

Next, we used MS and spectral counting to compare proteins present in the secretomes of the on- and off-state P. aeruginosa strains (Liu et al., 2004). Average spectral count (SC) values were used to identify whether each protein was differentially secreted between states. The results of our MS analyses are summarized in Table S1. Importantly, the total number of spectral counts was comparable between the on- and off-states in both replicates. A total of 371 proteins that met our filtering criteria were identified between replicate experiments (Tables S2). We divided the proteins into three groups: Category 1 (C1; Tables S3 and S4)—present in both the on- and off-states, Category 2 (C2; Table S5)—present only in the on-state, and Category 3 (C3; Table S6)—present only in the off-state. Overlap between the replicates was greatest among C1 proteins. A total of 314 C1 proteins were identified, of which 249 were shared between the replicates. A significant fraction of the C1 differences can be ascribed to the fact that 13% more proteins were identified in this category in Replicate 1 (R1) than in Replicate 2 (R2).

To assess the accuracy of the quantitative component of our datasets, we measured the distribution of SC ratios (on-state/off-state) within C1 proteins (FIG. 1D). Since we did not anticipate that the H1-T6SS should exhibit a global effect on the secretome, we were encouraged by the approximate split (50%±2 in both replicates) between those proteins that were up- versus down-regulated between the on- and off-states. Additionally, the change in average SCs between the states was low, and this value was similar in the replicates ([R1], 1.13±1.04; [R2], 1.15±0.90). Only 30 R1 and 33 R2 proteins yielded a SC ratio>2.

As expected, Hcp1 was over-represented in the on-state samples. Indeed, Hcp1 was the most differentially secreted protein in both datasets (SC ratio: [R1], 13; [R2], 17]) (FIG. 1D). The presence of Hcp1 in the secretome of off-state cells suggests a certain extent of cellular protein contamination within the preparations. This contamination is also evidenced by the predicted or known functions of many of the detected proteins (Tables S2-S4). The high abundance of Hcp1 (119 SC average) relative to the average protein abundance (10.9 SC) is likely another factor contributing to its detection in the off-state samples.

Next we analyzed C2 proteins—those observed only in the on-state. Similar numbers of these proteins were identified in R1 (19) and R2 (20), and five of these were found in both replicates (Table 1). The reproducibility of C2 versus C1 proteins is attributable to the difference in their average SCs; the average SC of C2 proteins was 2.6, versus 12 in C1. The C2 proteins identified in both R1 and R2 accounted for five of the six most abundant in C2-R1, and five of the ten most abundant in C2-R2. Each of these proteins lacked a secretion signal for known export pathways. The identity of these proteins and the biochemical validation of their secretion is the subject of subsequent sections.

The number and abundance of C3 proteins in both R1 and R2 was slightly lower than the corresponding C2 values. Nonetheless, we did identify three C3 proteins in common between R1 and R2 (Table 1). The occurrence of these proteins in the off-state is likely to reflect changes in gene regulation caused by modulation of the activity of the H1-T6SS that manifest in the secretome. Sequence analysis indicated that each of these proteins contains a predicted signal peptide (Emanuelsson et al., 2007).

### Two VgrG Proteins are Secreted by the H1-T6SS

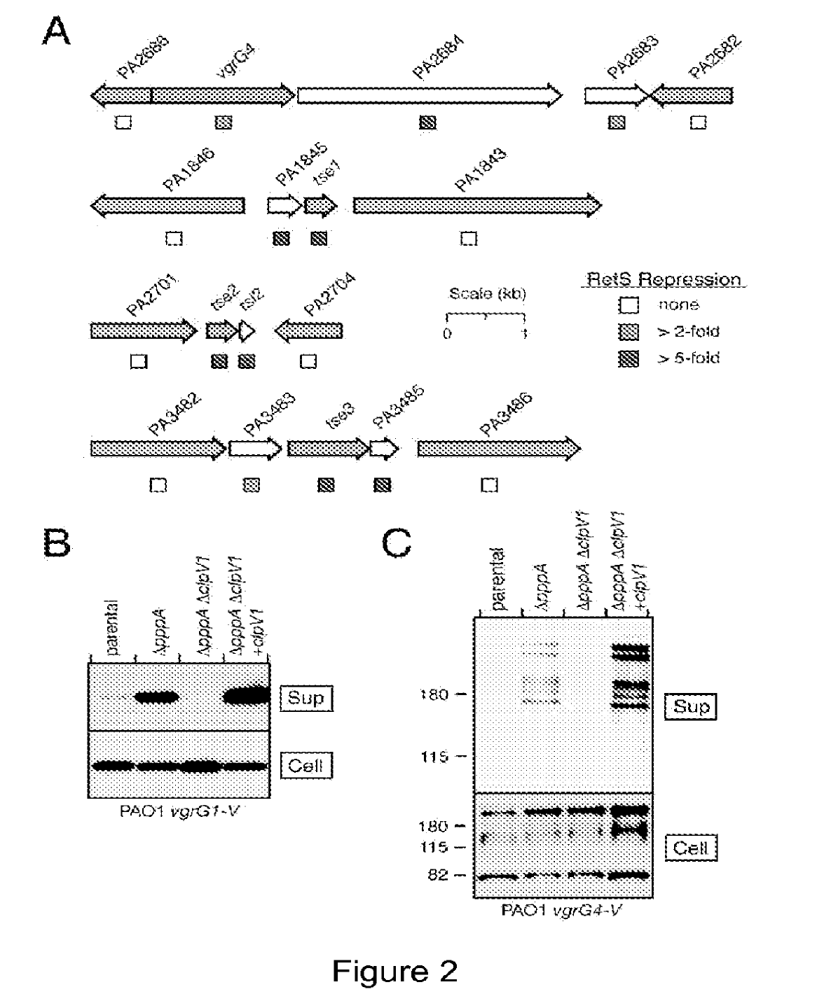

Two VgrG-family proteins, the products of open reading frames PA0091 and PA2685, were the most abundant C2 proteins in R1 and R2 (Table 1). Interestingly, earlier microarray work has shown that PA0091 and PA2685 are coordinately regulated with HSI-I by the RetS hybrid two-component sensor/response regulator protein, however the participation of these proteins in the H1-T6SS was not investigated (FIG. 2A) (Goodman et al., 2004; Laskowski and Kazmierczak, 2006; Zolfaghar et al., 2005). The PA0091 locus is located within HSI-I, while the PA2685 locus is found at an unlinked site that lacks other apparent T6S elements (FIGS. 1A and 2A). To remain consistent with previous nomenclature, these genes will henceforth be referred to as vgrG1 and vgrG4 (Mougous et al., 2006).

To confirm the MS results, we compared the localization of VgrG1 and VgrG4 in wild-type bacteria to strains containing the on-state (ΔpppA) and off-state (ΔclpV1) mutations. Consistent with our MS findings, Western blot analyses of cell and supernatant fractions in vgrG1-V and vgrG4-V backgrounds indicated that secretion of the proteins is strongly repressed by pppA and requires clpV1 (FIGS. 2B and 2C). These data show that the H1-T6SS exports at least two VgrG-family proteins. For reasons not yet understood, VgrG4-V migrated as two major bands in the cellular fraction and a large number of high molecular weight bands in the supernatant.

### Identification of Three H1-T6SS Substrates

The remaining C2 proteins identified in both R1 and R2 are proteins encoded by ORFs PA1844, PA2702, and PA3484. Interestingly, an earlier study identified the product of PA1844 as an immunogenic protein expressed by a P. aeruginosa clinical isolate (Wehmhoner et al., 2003). Bioinformatic analyses of the three proteins indicated that they do not share detectable sequence homology to each other or to proteins outside of P. aeruginosa. Each protein is encoded by an ORF that resides in a predicted two-gene operon with a second hypothetical ORF. Intriguingly, we noted that the three unlinked operons—like HSI-I (which includes vgrG1) and vgrG4—are negatively regulated by RetS (FIG. 2A).

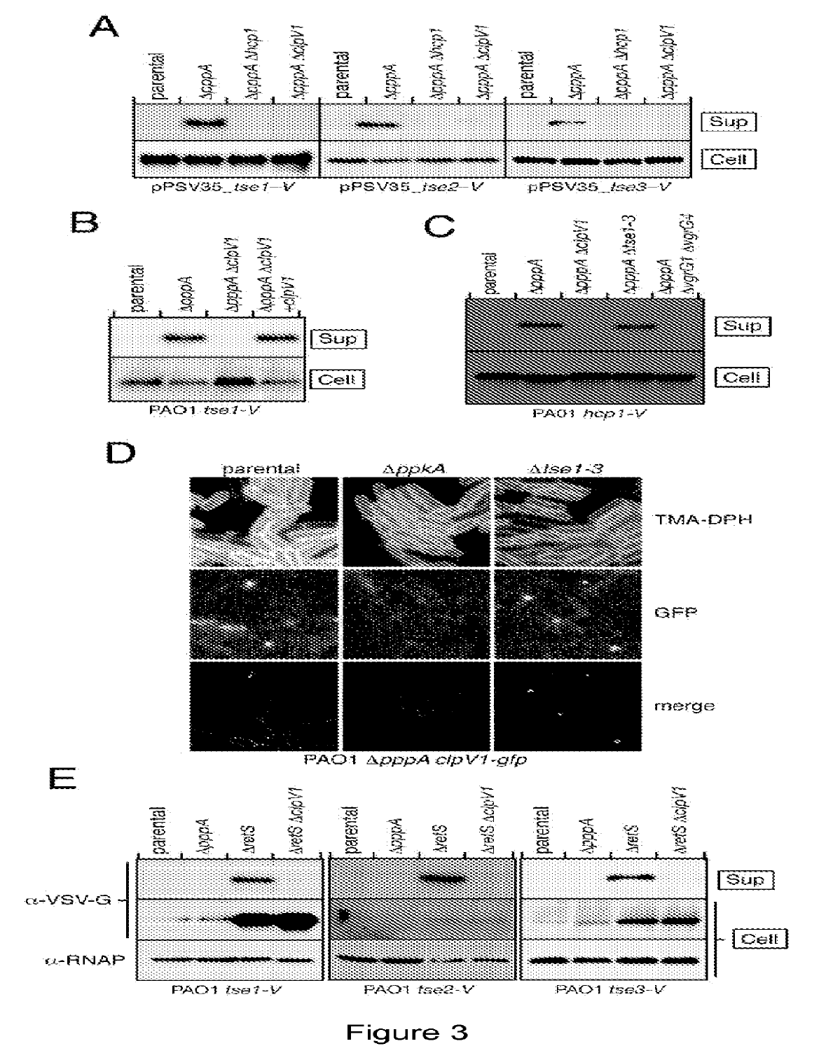

Based on our secretome analyses, we hypothesized that the proteins encoded by PA1844, PA2702, and PA3484, henceforth referred to Tse1-3, respectively, are substrates of the H1-T6SS. To test this, we analyzed the localization of the proteins when ectopically expressed in a diagnostic panel of P. aeruginosa strains. The secretion profile of each protein was similar in these strains; relative to the wild-type, ΔpppA displayed dramatically increased levels of secretion, and secretion levels were at or below wild-type levels in ΔpppA strains containing additional deletions in either hcp1 or clpV1 (FIG. 3A). Over-expression of the proteins was ruled out as a confounding factor, as the secretion profile of chromosomally-encoded Tse1-V in related backgrounds was similar to that of the ectopically-expressed protein (FIG. 3B). Finally, we complemented Tse1-V secretion in ΔpppA ΔclpV1 tse1-V with a plasmid expressing clpV1.

To further distinguish the Tse proteins as H1-T6SS substrates rather than structural components, we determined their influence on core functions of the T6 secretion apparatus. Fundamental to each studied T6SS is the ability to secrete an Hcp-related protein. In a systematic analysis, Hcp secretion was shown to require all predicted core T6SS components, including VgrG-family proteins (Pukatzki et al., 2007; Zheng and Leung, 2007). We generated a strain containing a deletion of all tse genes in the ΔpppA hcp1-V background and compared Hcp1 secretion in this strain to strains lacking both vgrG1 and vgrG4 or clpV1 in the same background. Western blot analysis revealed that Hcp1 secretion was abolished in both the ΔclpV1 and ΔvgrG1 ΔvgrG4 strains, however it was unaffected by tse deletion (FIG. 3C).

A multiprotein complex containing ClpV1 is essential for a functional T6S apparatus (Hsu et al., 2009). As a second indicator of H1-T6SS function, we used fluorescence microscopy to examine the formation of this complex in strains containing a chromosomal fusion of clpV1 to a sequence encoding the green fluorescent protein (clpV1-GFP) (Mougous et al., 2006). In line with the Hcp1 secretion result, the punctate appearance of ClpV1-GFP localization, which is indicative of proper apparatus assembly, was not dependent on the tse genes (FIG. 3D). On the other hand, deletion of ppkA, a gene required for assembly of the H1-T6S apparatus, disrupted ClpV1-GFP localization. Together, these findings provide evidence that the Tse proteins are substrates of H1-T6SS.

### Tse Secretion is Triggered by De-Repression of the Gac/Rsm Pathway

Earlier microarray experiments suggested that the tse genes are tightly repressed by RetS, a component of the Gac/Rsm signaling pathway (Lapouge et al., 2008). In this pathway, the activity of RetS and two other sensor kinase enzymes, LadS and GacS, converge to reciprocally regulate an overlapping group of acute and chronic virulence pathways in P. aeruginosa through the small RNA-binding protein RsmA (Brencic and Lory, 2009; Goodman et al., 2004; Ventre et al., 2006). To directly investigate the effect of the Gac/Rsm pathway on tse expression, we monitored the abundance of Tse proteins in the cell-associated and secreted fractions of strains containing the retS deletion. Our data showed that activation of the Gac/Rsm pathway dramatically elevates cellular Tse levels and triggers their export via the H1-T6SS (FIG. 3E). It is noteworthy that secretion of Tse proteins in ΔretS is far in excess of that observed in ΔpppA (FIG. 3E, compare ΔpppA and ΔretS).

Tsi2 is an Essential Protein that Protects P. aeruginosa from Tse2

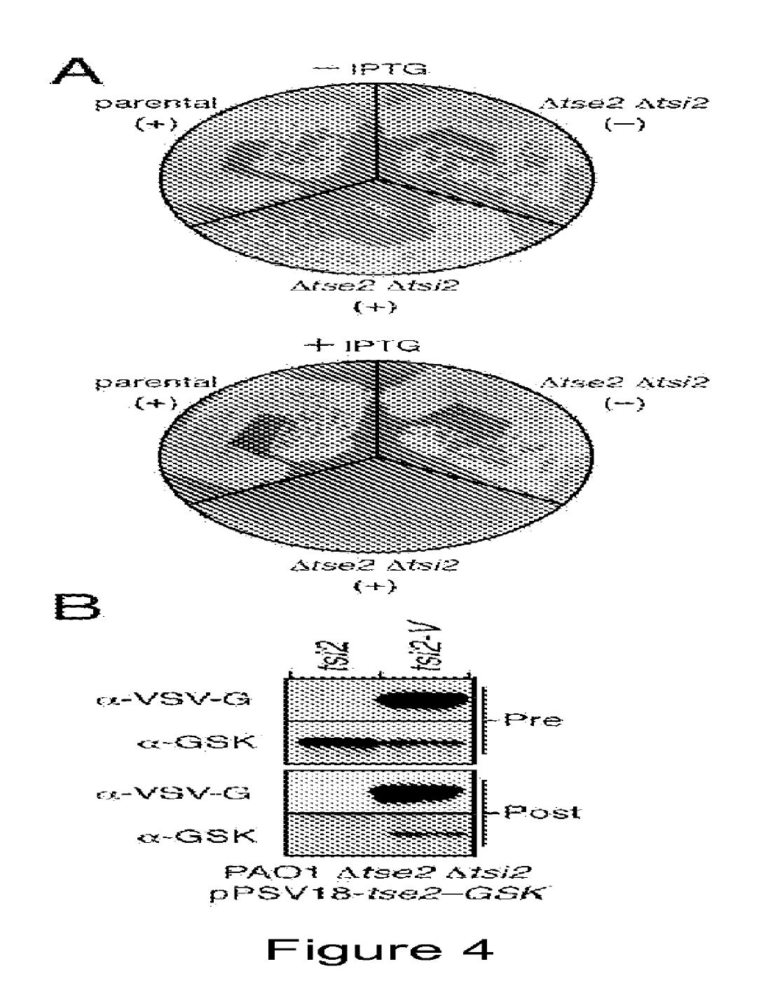

The lack of transposon insertions within the tse2/tsi2 locus in a published transposon insertion library of P. aeruginosa PAO1 suggested that these ORFs may be essential for viability of the organism (Jacobs et al., 2003). To test this possibility, we attempted to generate deletions of tse2 and tsi2. While a Δtse2 strain was readily constructed, tsi2 was refractory to several methods of deletion. Based on genetic context and co-regulation (FIG. 2A), we hypothesized that Tse2 and Tsi2 could interact functionally, and that the requirement for tsi2 could therefore depend on tse2. Success in simultaneous deletion of both genes confirmed this hypothesis (FIG. 4A).

Our findings implied that Tsi2 protects cells from Tse2. To probe this possibility further, we introduced tse2 to the Δtse2 Δtsi2 background. Induction of tse2 expression completely abrogated growth of Δtse2 Δtsi2, however it had only a mild effect on wild-type cells. These data demonstrate that tse2 encodes a toxic protein capable of inhibiting the growth of P. aeruginosa, and that tsi2 encodes a cognate immunity protein. We named Tsi2 based on this property (type VI secretion immunity protein 2).

Tsi2 could block the activity of Tse2 through a mechanism involving direct interaction of the proteins, or by an indirect mechanism wherein the proteins function antagonistically on a common pathway. To determine if Tse2 and Tsi2 physically interact, we conducted co-immunoprecipitation studies in P. aeruginosa. Tse2 was specifically identified in precipitate of Tsi2-V, indicative of a stable Tse2-Tsi2 complex (FIG. 4B). These data provide additional support for a functional interaction between Tse2 and Tsi2, and they suggest that the mechanism of Tsi2 inhibition of Tse2 is likely to involve physical association of the proteins.

### Intracellular Tse2 is Toxic to a Broad Spectrum of Prokaryotic and Eukaryotic Cells

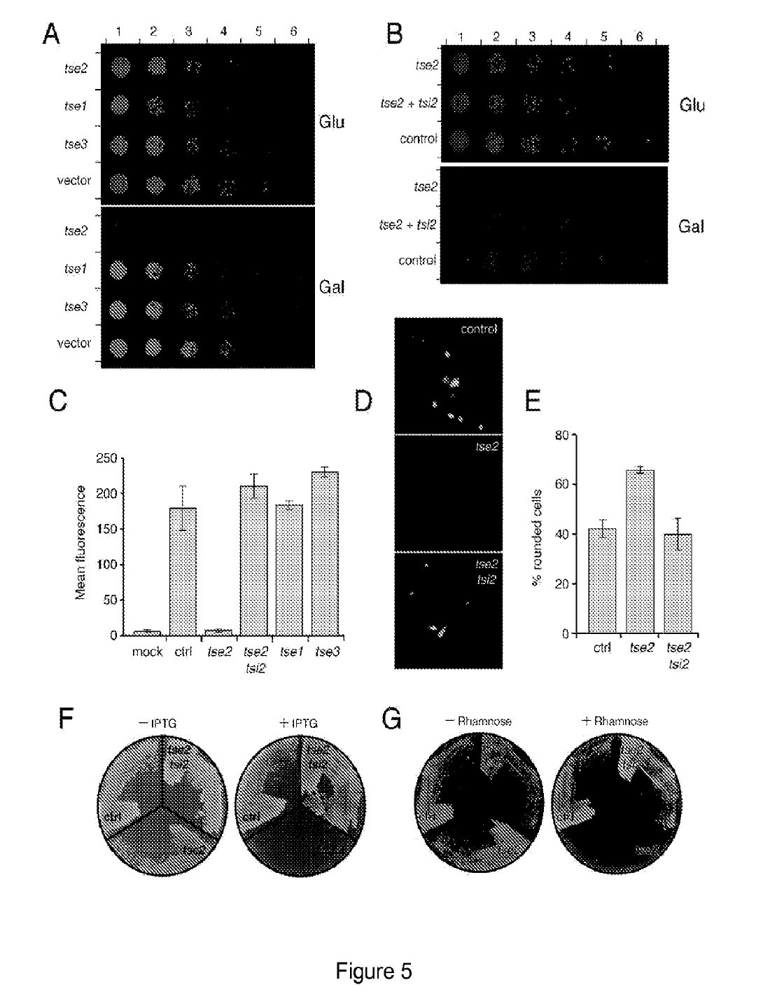

P. aeruginosa is widely dispersed in terrestrial and aquatic environments, and it is also an opportunistic pathogen with a diverse host range. As such, Tse2 exported from P. aeruginosa has the potential to interact with a range of organisms, including prokaryotes and eukaryotes. To investigate the organisms that Tse2 might target, we expressed tse2 in the cytoplasm of representative species from each domain. Two eukaryotic cells were chosen for our investigation, Saccharomyces cerevisiae and the HeLa human epithelial-derived cell line. Yeast were included primarily for diversity, however these organisms also interact with P. aeruginosa in assorted environments and could therefore represent a target of the toxin (Wargo and Hogan, 2006). S. cerevisiae cells were transformed with a galactose-inducible expression plasmid for each tse gene, or with an empty control plasmid (Mumberg et al., 1995). Relative to the other tse genes and the control, tse2 expression caused a dramatic decrease in observable colony forming units following 48 hrs of growth under inducing conditions (FIG. 5A). To address the specificity of Tse2 effects on S. cerevisiae, we next tested whether Tsi2 could block Tse2-mediated toxicity. Co-expression of tsi2 with tse2 restored viability to levels similar to the control strain (FIG. 5B). This result implies that the effects of Tse2 on S. cerevisiae are specific and that the toxin may act via a similar mechanism in bacteria and yeast. Our findings are consistent with an earlier screen for P. aeruginosa proteins toxic to yeast. Arnoldo et al. found Tse2 among nine P. aeruginosa proteins most toxic to S. cerevisiae within a library of 505 that included known virulence factors (Arnoldo et al., 2008).

The effects of Tse2 on a mammalian cell were probed using a reporter co-transfection assay in HeLa cells. Expression plasmids containing the tse genes were generated and mixed with a GFP reporter plasmid. Co-transfection of the reporter plasmid with tse1 and tse3 had no impact on GFP expression relative to the control; however, inclusion of the tse2 plasmid reduced GFP expression to background levels (FIGS. 5C and 5D). We also noted morphological differences between cells transfected with tse2 and control transfections, which was apparent in the fraction of rounded cells (FIG. 5E). These were specific effects of Tse2, as the inclusion of a tsi2 expression plasmid into the tse2/GFP reporter plasmid transfection restored GFP expression and lowered the fraction of rounded cells to the control. From these studies, we conclude that Tse2 has a deleterious effect on essential cellular processes in assorted eukaryotic cell types.

Next we asked whether Tse2 has activity in prokaryotes other than P. aeruginosa. We tested two organisms, Escherichia coli and Burkholderia thailandensis. Both organisms were transformed with plasmids engineered for inducible expression of either tse2, or as a control, both tse2 and tsi2. In each case, tse2 expression strongly inhibited growth and co-expression with tsi2 reversed this effect (FIGS. 5F and 5G). Taken together with the effects we observed in S. cerevisiae and HeLa cells, we conclude that Tse2 is a toxin that—when administered intracellularly—inhibits essential cellular processes in a broad spectrum of organisms.

P. aeruginosa can Target Bacterial, but not Eukaryotic Cells, with Tse2

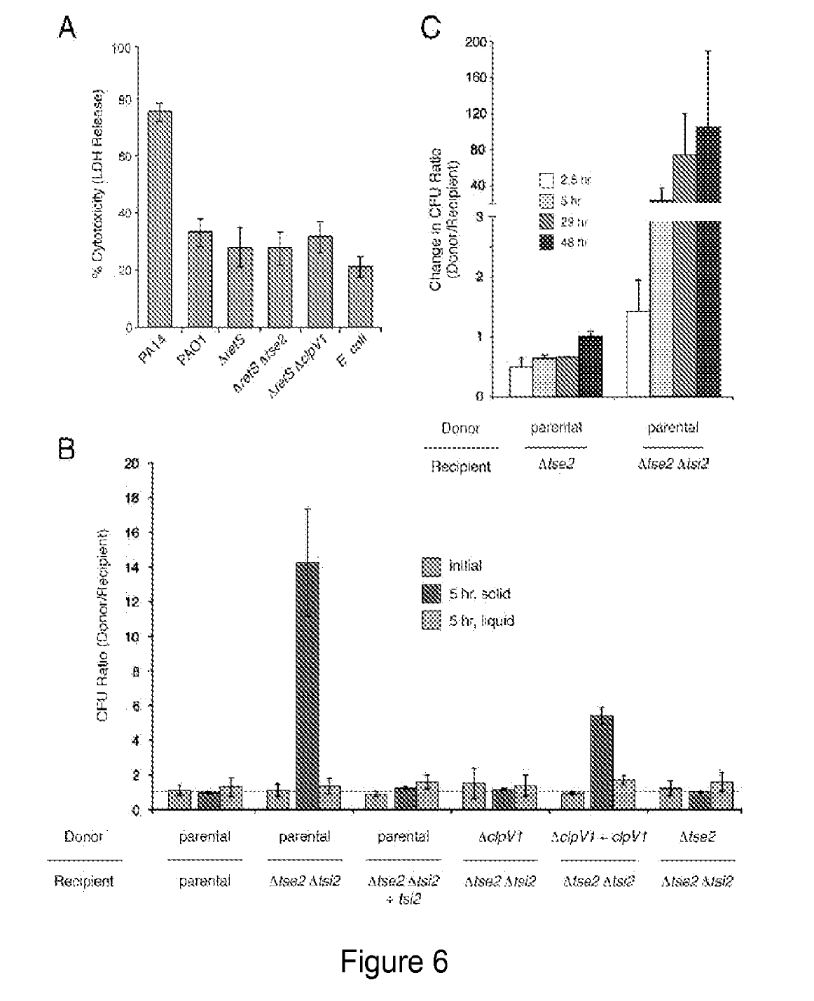

Since tse2 expression experiments indicated that the toxin could act on eukaryotes (FIG. 5A-E), we asked whether P. aeruginosa could target these cells with the H1-T6SS. We measured cytotoxicity toward HeLa and J774 cells for a panel of P. aeruginosa strains, including Tse2 hyper-secreting (ΔretS) and non-secreting backgrounds (ΔretS ΔclpV1). Under all conditions analyzed, we were unable to observe Tse2-promoted cytotoxicity or a morphological impact on the cells as was observed in transfection experiments (FIG. 6A and data not shown). Additionally, attempts to detect Tse2 or other Tse proteins in mammalian cell cytoplasm yielded no evidence of translocation (data not shown). We also investigated Tse2-dependent effects on yeast co-cultured with P. aeruginosa; again, no effect could be attributed to Tse2 (FIG. 51). Based on our data, we concluded that P. aeruginosa is unlikely to utilize Tse2 as a toxin against eukaryotic cells. This is in-line with results of earlier reports, which have shown that strains lacking retS are highly attenuated in acute virulence-related phenotypes, including macrophage and epithelial cell cytotoxicity (Goodman et al., 2004; Zolfaghar et al., 2005), and acute pneumonia and corneal infections in mice (Zolfaghar et al., 2006) (Laskowski et al., 2004).

The influence of intracellular tse2 expression on the growth of bacteria prompted us to next investigate whether its target could be another prokaryotic cell. To test this, we conducted a series of in vitro growth competition experiments with P. aeruginosa strains in the ΔretS background engineered with regard to their ability to produce, secrete, or resist Tse2. Competitions between these strains were conducted in liquid medium or following filtration onto a porous solid support. Neither production nor secretion of Tse2, nor immunity to the toxin, impacted the growth rates of competing strains in liquid medium (FIG. 6B). On the contrary, a striking proliferative advantage dependent on tse2 and tsi2 was observed when cells were grown on a solid support. In growth competition experiments between ΔretS and ΔretS Δtse2 Δtsi2, henceforth referred to as donor and recipients strains, respectively, donor cells were approximately 14-fold more abundant after 5 hours (FIG. 6B). This was entirely Tse2 mediated, as a deletion of tse2 from the donor strain, or the addition of tsi2 to the recipient strain, abrogated the growth advantage. Inactivation of clpV1 within the donor strain confirmed that the Tse2-mediated growth advantage requires a functional H1-T6SS (FIG. 6B). Importantly, the total proliferation of the donor remained constant in each experiment, indicating that Tse2 suppresses growth of the recipient strain.

In order to examine the extent to which Tse2 could facilitate a growth advantage, we conducted long-term competitions between strains with and without Tse2 immunity. The experiments were initiated with a donor-to-recipient cell ratio of approximately 10:1, raising the probability that each recipient cell will contact a donor cell. After 48 hours, the Tse2 donor strain displayed a remarkable 104-fold growth advantage relative to a recipient strain lacking immunity (FIG. 6C). These data conclusively demonstrate that the P. aeruginosa H1-T6SS can target Tse2 to another bacterial cell. The differences observed between competitions conducted in liquid medium versus on a solid support suggest that intimate donor-recipient cell contact is required. We have not directly demonstrated that Tse2 is translocated into recipient cell cytoplasm, however it is a likely explanation for our data given that cell contact is required and Tsi2 is a cytoplasmic immunity protein that physically interacts with the toxin (FIG. 4B).

### Discussion

The T6SS has been implicated in numerous, apparently disparate processes. With few exceptions, the mode-of-action of the secretion system in these processes is not known. Since the T6SS architecture appears highly conserved, we based our study on the supposition that the diverse activities of T6SSs, including T6SSs within a single organism, must be attributable to a diverse array of substrate proteins exported in a specific manner by each system. Our findings support this model; we identified three T6S substrates that lack orthologs outside of P. aeruginosa, and that specifically require the H1-T6SS for their export (FIGS. 1 and 3).

Bacterial genomes encode a large and diverse array of toxin-immunity protein (TI) systems (Gerdes et al., 2005). These can be important for plasmid maintenance, stress response, programmed cell death, cell-fate commitment, and defense against other bacteria. Tse2 differs from other TI toxins in that it is exported through a large, specialized secretion apparatus, while many TI system toxins are either not actively secreted, or they utilize the sec pathway (Riley and Wertz, 2002). This distinction implies that secretion through the T6S apparatus is required to target Tse2 to a relevant environment, cell, or subcellular compartment. Indeed, we have shown that targeting of Tse2 by the T6S apparatus is essential for its activity (FIG. 6).

We found that Tse2 is active against assorted bacteria and eukaryotic cells when expressed intracellularly (FIGS. 4 and 5). Despite this, we found no evidence that P. aeruginosa can target Tse2 to a eukaryotic cell, including mammalian cells of epithelial and macrophage origin (FIG. 6A and data not shown). Surprisingly, P. aeruginosa efficiently targeted the toxin to another bacterial cell (FIG. 6). These findings, combined with the following recent observations, provide support for the hypothesis that the T6SS can serve as an inter-bacterial interaction pathway. First, the secretion system is present and conserved in many non-pathogenic, solitary bacteria (Bingle et al., 2008; Boyer et al., 2009). Second, there is experimental evidence supporting an evolutionary relationship between extracellular components of the secretion apparatus and the tail proteins of bacteriophages T4 and (Ballister et al., 2008; Leiman et al., 2009; Pell et al., 2009; Pukatzki et al., 2007). Finally, two recent reports have implicated the conserved T6S component, VgrG, in inter-bacterial interactions. A bioinformatic analysis of Salmonella genomes identified a group of “evolved” VgrG proteins bearing C-terminal effector domains highly related to bacteria-targeting S-type pyocins, and a VgrG protein from Proteus mirabilis was shown to participate in an intra-species self/non-self recognition pathway (Blonde) et al., 2009; Gibbs et al., 2008).

It is also evident that in certain instances the T6SS has evolved to engage eukaryotic cells. In at least two reports, the T6S apparatus has been demonstrated to deliver a protein to a eukaryotic cell (Ma et al., 2009; Suarez et al., 2009). Moreover, the T6SSs of several pathogenic bacteria are major virulence factors (Bingle et al., 2008). Taken together with our findings, we posit that there are two broad groups of T6SSs, those that target bacteria and those that target eukaryotes. It is not possible at this time to rule out that a given T6SS may have dual specificity. However, our inability to detect the effects of Tse2 in an infection of a eukaryotic cell, and the fact that a Tse2 hyper-secreting strain is attenuated in animal models of acute infection (Laskowski et al., 2004; Zolfaghar et al., 2006), suggests that the T6S apparatus can be highly discriminatory. In this regard, it is instructive to consider other secretion systems that have evolved from inter-bacterial interaction pathways. The type IVA and type IVB secretion systems are postulated to have evolved from a bacterial conjugation system ((Burns, 2003; Christie et al., 2005; Lawley et al., 2003). These systems have become efficient at eukaryotic cell intoxication, however measurements indicate that substrate translocation into bacteria occurs at a frequency of only ˜1×10⁻⁶/donor cell (Luo and Isberg, 2004). In contrast, Tse2 targeting to bacteria by the H1-T6SS appears many orders of magnitude more efficient, as the donor strain in our assays is able to effectively suppress the net growth of an equal amount of recipient cells. The host adapted type IV secretion systems and the H1-T6SS represent two apparent extremes in the cellular targeting specificity of Gram-negative specialized secretion systems. Furthermore, they show that a high degree of discrimination can exist between pathways targeting eukaryotes and prokaryotes.

The physiologically relevant target bacteria of Tse2 and the H1-T6SS remains an open question. We have initiated studies to address the role of these factors in interspecies interactions, however we have not yet identified an effect. This may be because diffusible anti-bacterial molecules released by P. aeruginosa dominate the outcome of growth competitions performed under the conditions used in FIG. 6 (Hoffman et al., 2006; Kessler et al., 1993; Voggu et al., 2006). In future studies designed to allow free diffusion of these factors, and thereby more closely mimic a natural setting, their role may be mitigated. Interestingly, all sequenced P. aeruginosa strains appear to encode orthologs of tse2 and tsi2. Additionally, we found the genes universally present within a library of 44 randomly selected CF patient clinical isolates (Figure S2). Despite these findings, it remains possible that Tse2-mediated inter-P. aeruginosa interactions could be relevant in a natural context. For instance, it may not be simply the presence or absence of the toxin or its immunity protein, but rather the extent and manner in which these traits are expressed that decides the outcome of an interaction. In prior investigations of clinical isolates, we noted a high degree of heterogeneity in H1-T6SS activation, as judged by Hcp1 secretion levels (Mougous et al., 2006; Mougous et al., 2007). The wild-type strain used in the current study does not secrete Hcp1, and in this background the H1-T6SS does not provide a growth advantage against an immunity-deficient strain (data not shown). However, the H1-T6SS activation state of many clinical isolates resembles the ΔretS background, and therefore these strains are likely capable of using Tse2 in competition with other bacteria. In this context, it is intriguing that tse and HSI-I expression are subject to strict regulation by the Gac/Rsm pathway (FIG. 3E). Since this pathway responds to bacterial signals, including those of the sensing strain and other Pseudomonads (Lapouge et al., 2008), it is conceivable that cell-cell recognition could be an important aspect of Tse2 production and resistance.

The cell-cell contact requirement of H1-T6SS-dependent delivery of Tse2 suggest that the system could play an important role in scenarios involving relatively immobile cells, such as cells encased in a biofilm. The polyclonal and polymicrobial lung infections of patients with CF, wherein the bacteria are thought to reside within biofilm-like structures, is one setting where Tse2 could provide a fitness advantage to P. aeruginosa (Sibley et al., 2006; Singh et al., 2000). Intriguingly, P. aeruginosa is particularly adept at adapting to and competing in this environment, and studies have shown that it can even displace preexisting bacteria (D'Argenio et al., 2007; Deretic et al., 1995; Hoffman et al., 2006; Nguyen and Singh, 2006) (Foundation, 2007). If Tse2 does play a key role in the fitness of P. aeruginosa in a CF infection, this could explain the elevated expression and activation of the H1-T6SS observed in isolates from CF patients (Mougous et al., 2006; Mougous et al., 2007; Starkey et al., 2009; Yahr, 2006).

### Experimental Procedures

### Bacterial Strains, Plasmids and Growth Conditions

The P. aeruginosa strains used in this study were derived from the sequenced strain PAO1 (Stover et al., 2000). P. aeruginosa were grown on Luria-Bertani (LB) medium at 37° C. supplemented with 30 μg ml⁻¹ gentamicin, 300 g ml⁻¹ carbenicillin, 25 μg ml⁻¹ irgasan, 5% w/v sucrose, 0.5 mM IPTG and 40 μg ml⁻¹ X-gal (5-bromo-4-chloro-3-indolyl β-D-galactopyranoside) as required. Burkholderia thailandensis E264 and Escherichia coli BL21 were grown on LB medium containing 200 μg ml⁻¹ trimethoprim, 50 μg ml⁻¹ kanamycin, 0.2% w/v glucose, 0.2% w/v rhamnose and 0.5 mM IPTG as required. E. coli SM10 used for conjugation with P. aeruginosa was grown in LB medium containing 15 g ml⁻¹ gentamicin. Plasmids used for inducible expression include pPSV35, pPSV35CV, and pSW196 for P. aeruginosa (Baynham et al., 2006; Hsu et al., 2009; Rietsch et al., 2005), pET29b (Novagen) for E. colit, pSCrhaB2 (Cardona and Valvano, 2005) for B. thailandensis, and p426-GAL-L and p423-GAL-L for S. cerevisiae (Mumberg et al., 1995). Chromosomal fusions and gene deletions were generated as described previously (Mougous et al., 2006; Rietsch et al., 2005). See Supplemental Experimental Procedures for specific cloning procedures.

### Secretome Preparation

Cells were grown to optical density 600 nm (OD₆₀₀) 1.0 in Vogel-Bonner minimal medium containing 19 mM amino acids as defined in synthetic CF sputum medium (Palmer et al., 2007). The presence of amino acids was required for H1-T6SS activity (data not shown). Proteins were prepared as described previously (Wehmhoner et al., 2003).

### Mass Spectrometry

Precipitated proteins were suspended in 100 μl of 6 M urea in 50 mM NH₄HCO₃, reduced and alkylated with dithiotreitol and iodoactamide, respectively, and digested with trypsin (50:1 protein:trypsin ratio). The resultant peptides were desalted with Vydac C18 columns (The Nest Group) following the manufacturer's protocol. Samples were dried to 5 μL, resuspended in 0.1% formic acid/5% acetonitrile and analyzed on an LTQ-Orbitrap mass spectrometer (Thermo Fisher) in triplicate. Data was searched using Sequest (Eng et al., 1994) and validated with Peptide/Protein Prophet (Keller et al., 2002). The relative abundance for identified proteins was calculated using spectral counting (Liu et al., 2004). See Supplemental Experimental Procedures additional MS procedures.

### Preparation of Proteins and Western Blotting

Cell-associated and supernatant samples were prepared as described previously (Hsu et al., 2009). Western blotting was performed as described previously (Mougous et al., 2006), with the exception that detection of the Tse proteins required primary antibody incubation in 5% BSA in Tris-buffered saline containing 0.05% v/v Tween 20 (TBST). The GSK tag was detected using -GSK (Cell Signaling Technologies).

### Immunoprecipitation

Cells grown in appropriate additives were harvested at mid-log phase by centrifugation (6,000×g, 3 min) at 4° C. and resuspended in 10 ml of Buffer 1 (200 mM NaCl, 20 mM Tris pH 7.5, 5% glycerol, 2 mM dithiothreitol, 0.1% triton) containing protease inhibitors (Sigma) and lysozyme (0.2 mg ml⁻¹). Cells were disrupted by sonication and the resulting lysate was clarified by centrifugation (25,000×g, 30 min) at 4° C. A sample of the supernatant material was removed (Pre) and the remainder was incubated with 100 l of -VSV-G agarose beads (Sigma) for 2 hours at 4° C. for. Beads were washed three times with 15 ml of Buffer 1 and pelleted by centrifugation. Proteins were eluted with SDS-PAGE loading buffer.

### Fluorescence Microscopy

Mid-log phase cultures were harvested by centrifugation (6,000×g, 3 min), washed with phosphate-buffered saline (PBS), and resuspended to OD₆₀₀ 5 with PBS containing 0.5 mM TMA-DPH (Molecular Probes). Microscopy was performed as described previously (Hsu et al., 2009). All images shown were manipulated identically.

### Yeast Toxicity Assays

Saccharomyces cerevisiae BY4742 (MATα his3Δ1 leu2Δ0 lys2Δ0 ura3Δ0) was transformed with p426-GAL-L containing tse1, tse2, tse3, or the empty vector, and grown o/n in SC−Ura+2% glucose (Mumberg et al., 1995). Cultures were resuspended to OD₆₀₀ 1.0 with water and serially diluted fivefold onto SC−Ura+2% glucose agar or SC−Ura+2% galactose+2% raffinose agar. Plates were incubated at 30° C. for 2 days before being photographed. The tsi2 gene was cloned into p423-GAL-L and transformed into S. cerevisiae BY4742 harboring the p426-GAL-L plasmid. Cultures were grown o/n in SC−Ura−His+2% glucose.

### Growth Competition Assays

Overnight cultures were mixed at the appropriate donor-to-recipient ratio to a total density of approximately 1.0×10⁸ CFU/ml in 5 ml LB medium. In each experiment, either the donor or recipient strain contained lacZ inserted at the neutral phage attachment site (Vance et al., 2005). This gene had no effect on competition outcome. Co-cultures were either filtered onto a 47-mm 0.2 μm nitrocellulose membrane (Nalgene) and placed onto LB agar or were inoculated 1:100 into 2 ml LB (containing 0.4% w/v L-arabinose, if required), and were incubated at 37° C. with shaking. Filter-grown cells were resuspended in LB medium and plated on LB agar containing X-gal.

### Cell Culture and Infection Assays

HeLa cells were cultured and maintained in Dulbecco's modified eagle medium (DMEM, Invitrogen) supplemented with 10% Fetal Bovine Serum (FBS) and 100 μg ml⁻¹ penicillin or streptomycin as required. Incubations were performed at 37° C. in the presence of 5% CO₂. Infection assays were carried out using cells seeded in 96-well plates at a density of 2.0×10⁴ cells/well. Following o/n incubation, wells were washed in 1× Hank's balanced salt solution and DMEM lacking sodium pyruvate and antibiotics was added. Bacterial inoculum was added to wells at a multiplicity of infection of 50 from cultures of OD₆₀₀ 1.0. Following incubation for 5 hours, the percent cytotoxicity was measured using the CytoTox-One assay (Promega).

### Transient Transfection, Cell Rounding Assays, and Flow Cytometric Analysis

HeLa cells were seeded in 24-well flat bottom plates at a density of 2.0×10⁵ cells/well and incubated o/n in DMEM supplemented with 10% FBS. Reporter co-transfection experiments were performed using Lipofectamine according to the manufacturer's protocol. Total amounts of transfected DNA were normalized using equal quantities of the GFP reporter plasmid (empty pEGFP-N1 (Clonetech)), one of the tse expression plasmids (pEGFP-N1-derived), and either a non-specific plasmid or the tsi2 expression plasmid where indicated. Cell rounding was quantified manually using phase-contrast images from three random fields acquired at 40× magnification. Prior to flow cytometry, HeLa cells were washed two times and resuspended in 1×PBS supplemented with 0.75% FBS. Analysis was performed on a BD FACscan2 cell analyzer and mean GFP intensities were calculated using FlowJo 7.5 software (Tree Star, Inc.).

### EXAMPLE 2

Tse2 and Tsi2 mutants were generated and tested for cytotoxic activity and preservation of immunity to Tse2 cytotoxicity, respectively.

Truncation mutants listed in Table 1 were tested for (a) toxicity as judged by ectopic expression of allele in P. aeruginosa PAO1 Δtse2 Δtsi2., (b) expression as determined by -VSV-G Western blot, and (c) secretion determined by presence of indicated protein in concentrated supernatants prepared from PAO1 ΔretS Δtse2 versus PAO1 ΔretS Δtse2 ΔclpV1. The mutants listed in Table 1 are based on the P. aeruginosa PAO1 sequence (SEQ ID NO:2). All truncations were fused at their C-terminus to the VSV-G epitope.

TABLE 1
Toxicity and secretion via T6S of Tse2 truncation mutants.

| Tse2 residues | | | |
|---|---|---|---|
| present¹ | Toxicity² | Expression³ | Secretion⁴ |
| 7-158 | + | + | + |
| 10-158 | − | +− | − |
| 13-158 | − | +− | − |
| 16-158 | − | +− | − |
| 19-158 | − | +− | − |
| 22-158 | − | +− | − |
| 32-158 | − | + | − |
| 44-158 | − | + | − |
| 1-120 | − | + | − |
| 1-125 | − | + | − |
| 1-129 | − | + | − |
| 1-155 | + | + | + |

The lack of toxicity observed for those alleles that did not express fully (+/−) could be attributed to expression levels. The data presented in Table 1 show that Tse2 residues 1-6 and 156-158 are not required for toxicity.

A variety of Tse2 point mutants (Table 2) were also generated by Quikchange mutagenesis in the pPSV35-CV vector (see Hsu and Mougous, 2009 for plasmid reference). Toxicity, expression, and secretion were assessed as for the truncation mutants in Table 1.

TABLE 2
Toxicity and secretion via T6S of Tse2 point mutants.

| Tse2 amino acid | | | |
|---|---|---|---|
| substitution(s) | Toxicity | Expression | Secretion |
| S9A L10A | + | + | N/D |
| R60A | +− | + | + |
| T79A S80A | − | + | + |
| R89AR90A | − | N/D | N/D |
| Q119A | + | + | + |
| KP129-130AA | +− | + | − |
| QL139-140AA | + | + | N/D |
| RR149-150AA | + | + | N/D |

We next generated a series of Tsi2 mutants and tested for Tse2-immunity properties. Immunity was determined by ectopic expression of the indicated allele in P. aeruginosa Δtse2 Δtsi2. Growth of the strain indicates Tsi2 provides immunity, as Tse2 is co-expressed. Numbering of the Ts12 sequence is relative to the Tsi2 sequence of SEQ ID NO:4.

TABLE 3
Tsi2 mutants and associated Tse2-immunity properties.

| | Immu- | | Immu- |
|---|---|---|---|
| Mutants | nity | Mutants | nity |
| pET29-Tse2-Tsi2-cv (wild- | + | pET29-Tse2-Tsi2-D30A- | + |
| type) | | cv | |
| pET29-Tse2-Tsi2-alpha-cv | − | pET29-Tse2-Tsi2-Q32A- | + |
| | | cv | |
| pET29-Tse2-Tsi2-N2A-cv | + | pET29-Tse2-Tsi2-N33A- | + |
| | | cv | |
| pET29-Tse2-Tsi2-K4A-cv | + | pET29-Tse2-Tsi2- | + |
| | | E36Acv | |
| pET29-Tse2-Tsi2-Q6A-cv | + | pET29-Tse2-Tsi2-E38A- | + |
| | | cv | |
| pET29-Tse2-Tsi2-T7A-cv | + | pET29-Tse2-Tsi2-Q39A- | + |
| | | cv | |
| pET29-Tse2-Tsi2-L8A-cv | + | pET29-Tse2-Tsi2-Y44A- | + |
| | | cv | |
| pET29-Tse2-Tsi2-Q13A-cv | + | pET29-Tse2-Tsi2-D45A- | + |
| | | cv | |
| pET29-Tse2-Tsi2-R18A-cv | + | pET29-Tse2-Tsi2-D49A- | + |
| | | cv | |
| pET29-Tse2-Tsi2-R20A-cv | + | pET29-Tse2-Tsi2-D50A- | + |
| | | cv | |
| pET29-Tse2-Tsi2-E21A-cv | + | pET29-Tse2-Tsi2-K52A- | + |
| | | cv | |
| pET29-Tse2-Tsi2-Q25A-cv | + | pET29-Tse2-Tsi2-E56A- | + |
| | | cv | |
| pET29-Tse2-Tsi2-Q27A-cv | + | pET29-Tse2-Tsi2-Q57A- | + |
| | | cv | |
| pET29-Tse2-Tsi2-N28A-cv | + | pET29-Tse2-Tsi2-Q61A- | + |
| | | cv | |
| pET29-Tse2-Tsi2-D29A-cv | + | pET29-Tse2-Tsi2-A47Q- | +− |
| | | cv | |
| pET29-Tse2-Tsi2-V10Q-cv | + | pET29-Tse2-Tsi2-A11Q- | +− |
| | | cv | |
| pET29-Tse2-Tsi2-C14Q-cv | + | pET29-Tse2-Tsi2-V42Q- | + |
| | | cv | |
| pET29-Tse2-Tsi2-L46Q-cv | + | pET29-Tse2-Tsi2-1-59- | + |
| | | cv | |

The data presented in Table 3 show that mutations at virtually all positions retained tse2 immunity, demonstrating that Tsi2 is resilient and its interactions are robust. Truncation studies (not shown) demonstrated that residues 60-77 of Tsi2 can be removed while retaining its Tse2 immunity activity.

### EXAMPLE 3

### Creation of Vectors with the Tse2 Gene

The plasmid containing Tse2 can be constructed by cloning the complete Tse2 gene into any appropriate vector, as is well known in the art. The techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

Appropriate vectors may be obtained from, for example, Vector Laboratories Inc.; Promega; Novagen; New England Biolabs; Clontech; Roche; Pharmacia; EpiCenter; OriGenes Technologies Inc.; Stratagene; Perkin Elmer; Pharmingen; and Invitrogen Corp., Carlsbad, Calif. Such vectors may then for example be used for cloning or subcloning nucleic acid molecules of interest. General classes of vectors of particular interest include prokaryotic and/or eukaryotic cloning vectors, Expression Vectors, fusion vectors, two-hybrid or reverse two-hybrid vectors, shuttle vectors for use in different hosts, mutagenesis vectors, transcription vectors, and the like.

Once the appropriate plasmid vector is chosen, PCR can be used to amplify the Tse2 gene by designing appropriate primers for the DNA sequence. The PCR primers can be designed with restriction sites or recombination sites to facilitate cloning into the desired vector backbone. All recombination sites, restriction sites, other death genes, promoters, and other plasmid DNA elements can be amplified by PCR using the appropriate primer pairs as is well known in the art. The embodiments described herein depict the various arrangements of these plasmid DNA elements, and creation of such plasmid vectors is well within the ability of one of ordinary skill in the art.

### EXAMPLE 4

### Creation of Vectors with the Tsi2 Gene

The plasmid containing Tsi2 can be constructed by cloning the complete Tsi2 gene into any appropriate vector, as is well known in the art. The techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

Appropriate vectors may be obtained from, for example, Vector Laboratories Inc.; Promega; Novagen; New England Biolabs; Clontech; Roche; Pharmacia; EpiCenter; OriGenes Technologies Inc.; Stratagene; Perkin Elmer; Pharmingen; and Invitrogen Corp., Carlsbad, Calif. Such vectors may then for example be used for cloning or subcloning nucleic acid molecules of interest. General classes of vectors of particular interest include prokaryotic and/or eukaryotic cloning vectors, Expression Vectors, fusion vectors, two-hybrid or reverse two-hybrid vectors, shuttle vectors for use in different hosts, mutagenesis vectors, transcription vectors, and the like.

Once the appropriate plasmid vector is chosen, PCR can be used to amplify the Tsi2 gene by designing appropriate primers for the DNA sequence. The PCR primers can be designed with restriction sites or recombination sites to facilitate cloning into the desired vector backbone. All recombination sites, restriction sites, other death genes, promoters, and other plasmid DNA elements can be amplified by PCR using the appropriate primer pairs as is well known in the art. The embodiments described herein depict the various arrangements of these plasmid DNA elements, and creation of such plasmid vectors is well within the ability of one of ordinary skill in the art.

### EXAMPLE 5

### Creation of Linear Vectors Resistant to Recircularization

In one example, the Tse2 or Tsi2 vectors are linearized by HindIII, AccI, or other restriction digestion, which results in an overhang compatible with topoisomerase cloning, as is known in the art. Alternatively, the vectors can be prepared to have blunt or other customized overhangs at the ends of the linear vectors. As described in the literature, the ends of the vector can be covalently bound to topoisomerase I to facilitate cloning, such that a desired DNA fragment can be incubated along with the modified linearized Tse2 or Tsi2 vector, resulting in the DNA fragment entering the Tse2 or Tsi2 vector at the site of the restriction digestion.

In another example, the linearized Tse2 or Tsi2 vectors are treated with a dephosphorylating enzyme, such as alkaline phosphatase or an equivalent. This treatment reduces the likelihood that the Tse2 or Tsi2 vector will recircularize without incorporating the DNA fragment of interest, and thus increases positive cloning efficiency. One of ordinary skill in the art can use any other modifications to the vectors which will result in increased efficiency of production of vectors with the DNA fragment of interest.

### EXAMPLE 6

### Creation of Cell Lines Expressing Tse2

Any cell type can be used to express the vectors created herein, including both bacterial and eukaryotic cells. In many examples, the vectors will be transformed into E. coli and positive clones selected for accordingly. However, the vectors can be transfected into eukaryotic cells as well, such as CHO cells, HeLa cells, fibroblasts, or any other cell type desired by one of ordinary skill in the art.

Once the desired recombinant vector is created, the cells are transformed or transfected using electroporation, liposomes, calcium phosphate, or other techniques well known to one of ordinary skill in the art. In one example, the Tse2 gene is under the transcriptional control of an inducible promoter, such as the lac promoter, such that the Tse2 gene is not constitutively expressed in the cell line. Successful chromosomal integration can be selected for by using a second antibiotic resistance gene, such as chloramphenicol, which may or may not be found on the same plasmid containing the Tse2 gene. Any other selection markers can be used by one of ordinary skill in the art depending on the design of the research experiment.

### EXAMPLE 7

### Positive Selection of Tsi2-Containing Plasmids

To select for Tsi2-containing plasmids, the Tsi2 gene is included on a vector which will, when expressed, confer immunity to a cell which is expressing Tse2. The cells expressing Tse2 are created as described herein. In a cell line which is expressing Tse2 in the absence of Tsi2, the cells will not survive. Any of the vectors containing the Tsi2 gene or gene fragment described in the embodiments herein can be used for selection of positive clones containing the DNA fragment of interest.

If a Tse2-expressing cell receives the vector which expresses the Tsi2 gene, that prokaryotic or eukaryotic cell will survive, while such cells that do not express the Tsi2 gene will not survive. As noted in the examples herein, without intending to be bound to any particular mechanism, the mechanism of Tsi2 inhibition of Tse2 is likely to involve physical association of the proteins. In this selection example, the surviving cells will contain the plasmid with the DNA fragment of interest, along with Tsi2. If the plasmid containing the DNA fragment of interest is absent, the cells will die and will not be selected.

In another example, the vector containing a Tsi2 gene is used for selection of positive clones containing the DNA fragment of interest. Cells expressing the Tsi2 gene also contain the DNA fragment of interest on the vector. In this method, the Tsi2 gene can be used as a marker for a desired recombination or ligation event.

In another example, a vector containing a Tsi2 gene flanked by one or more recombination sites gene is used for selection of positive clones containing the DNA fragment of interest. The DNA fragment of interest is inserted into a site on the vector, such that the fragment does not disrupt the Tsi2 gene but is contained within the recombination sites. In another example, a topoisomerase or TA site is included within the flanking sites, but outside the Tsi2 gene, to facilitate DNA fragment insertion. The vector containing the DNA fragment of interest is then combined with a second vector containing matching recombination sites, such that a positive recombination event will move the DNA fragment of interest and the Tsi2 gene into the new vector, which can then be selected for survival in cells expressing Tse2, as described herein.

In another example, the vector containing the Tsi2 gene flanked by one or more restriction sites is used for selection of positive clones containing the DNA fragment of interest. The DNA fragment of interest is inserted into a site on the vector, such that the fragment does not disrupt the Tsi2 gene but is contained within the restriction sites. The vector containing the DNA fragment of interest and a second cloning vector are then digested with one or more restriction enzymes, followed by a ligation reaction. A positive ligation event will move the DNA fragment of interest and the Tsi2 gene into the second cloning vector, which can then be selected for survival in cells expressing Tse2.

In one example, the vector comprising a Tsi2 gene in an inactive form, such as a truncated form, is used for selection of positive clones containing the DNA fragment of interest. This vector can be used, for example, in methods for rescuing the activity of the Tsi2 gene such that vectors which contain a functional Tsi2 gene also contain the DNA fragment of interest (as described herein). The functional Tsi2 can be rescued by recombination, integration, or other events or reactions as described herein. Vectors can be readily designed for the particular experiment by one of ordinary skill in the art.

In another example, a vector containing the Tsi2 locus, but split into two parts on the same plasmid, is used for selection of positive clones containing the DNA fragment of interest. A fully functional Tsi2 would assemble through homologous recombination or ligation event, such that only the prokaryotic or eukaryotic cells containing a recombinant plasmid containing the DNA fragment of interest, with a functional Tsi2 can survive transformation.

### EXAMPLE 8

### Negative Selection of Tse2 Plasmids

The Tse2 recombinant vectors can be used in negative selection in order to enhance the efficiency of production of plasmids containing the desired DNA fragment of interest. In one example, the vector comprising one or more unique restriction enzyme recognition sites, wherein cloning of a nucleic acid insert into the one or more unique restriction enzyme recognition sites disrupts expression of Tse2, can be used to exclude vectors that do not contain the DNA fragment of interest. The vectors of this embodiment can be used as cloning vehicles, since cloning of an insert into the one or more restriction sites in the vector interrupts Tse2 expression and provide an easily selectable marker—cells with vectors containing no insert have their growth inhibited by Tse2 expression (so long as they do not endogenously express an antidote to Tse2), and those with inserts do not. In one preferred embodiment, one or more unique restriction sites are engineered into the coding region for Tse2 using techniques well known to those of skill in the art, such that cloning an insert into the restriction site disrupts the coding region for Tse2. In this embodiment, the restriction sites can be engineered into the coding region to result in silent nucleotide changes, or may result in one or more changes in the amino acid sequence of Tse2, so long as the encoded Tse2 protein retains cytotoxic activity. Alternatively, the one or more unique restriction sites may be located in regulatory regions such that cloning of an insert would disrupt expression of Tse2 from the vector. Design and synthesis of nucleic acid sequences and preparation of vectors comprising such sequences is well within the level of skill in the art.

The Tse2 recombinant vectors can also be used in negative selection, such as for example using the Gateway® Cloning System described herein. Any of the vectors described in the embodiments herein can be used to exclude vectors that do not contain the DNA fragment of interest, such that a functional Tse2 gene indicates a vector which is lacking the DNA fragment of interest.

In this example, the vector containing a Tse2 gene flanked by one or more restriction enzyme sites or recombination sites can be used to exclude vectors that do not contain the DNA fragment of interest. Recombination sites include, but are not limited to, attB, attP, attL, and attR. This vector is designed such that the DNA fragment of interest (such as, for example, a PCR product) will replace the Tse2 located between the two flanking sites. If the DNA fragment of interest is present in the vector, the cells containing the vector survive, as the Tse2 gene will no longer be present on the desired recombinant vector. If the gene of interest is not present, the Tse2 gene will prevent survival of the cell carrying the undesired vector. Thus, only cells containing positive clones with the DNA fragment of interest will be viable, and easily selected for.

In another example, the vector containing a dual selection cassette, wherein the vector comprises a first gene encoding Tse2, and a second gene encoding a second selectable marker, such as an antibiotic resistance gene or a second “death” gene encoding a second toxic protein, can be used to exclude vectors that do not contain the DNA fragment of interest. The antibiotic resistance gene can be selected from either bacterial or eukaryotic genes, and can confer resistance to ampicillin, kanamycin, tetracycline, cloramphenicol, and others known in the art. The second death gene can be any suitable death gene, including but not limited to, rpsL, tetAR, pheS, thyA, lacY, gata-1, ccdB, and sacB. The second death gene can also be selected from either prokaryotic or eukaryotic toxic genes. This dual selection cassette is flanked by at least one restriction site or recombination site, such that the DNA fragment of interest will replace the dual selection cassette located between the two sites in the desired recombination or ligation event. If the DNA fragment of interest is present, the cells containing the vector survive, as the Tse2 gene will no longer be present on the desired recombinant vector. If the gene of interest is not present, the vector will still contain the Tse2 gene and will prevent survival of the cell carrying the undesired vector. This dual selection cassette can thus be used for any double negative selection strategy as desired by one of ordinary skill in the art. In one embodiment, the Tse2 gene double negative selection strategy is used when use of multiple antibiotics is not be compatible with the particular selection design.

In another example, the vector containing a dual selection cassette comprising the Tse2 gene as well as a cloramphenicol resistance gene under control of at least one promoter, can be used to exclude vectors that do not contain the DNA fragment of interest. The vector is cut using restriction enzymes both upstream and downstream of the dual selection cassette. Optionally, the linearized vector can be gel purified to remove the excised dual selection cassette DNA from the reaction. DNA containing the DNA fragment of interest and appropriate restriction enzyme sites, such as a PCR product, is then combined with the linearized vector in a ligation reaction. Positive clones will be chloramphenicol sensitive and viable (Tse2 negative), due to the replacement of the dual selection cassette with the DNA fragment of interest.

In another example, the vector containing at least one recombination site within the Tse2 gene or corresponding regulatory element (e.g. promoter or enhancer), such that a desired recombination event will disrupt the expression of the Tse2 gene from the vector, can be used to exclude vectors that do not contain the DNA fragment of interest. The location of the recombination site should be chosen such that if the desired recombination event occurs, the resulting Tse2 gene will be inactive and the cell containing the desired vector will survive. If the desired recombination event does not occur, the Tse2 gene will remain intact and the cell containing the undesired vector will not survive.

In another example, the vector contains at least one restriction enzyme site within the Tse2 gene or corresponding regulatory element (e.g. promoter or enhancer), which is used to exclude vectors that do not contain the DNA fragment of interest, such that an undesired ligation event will produce an intact and functional Tse2 gene, which will result in the death of the cell containing the undesired vector.

In another example, the Tse2 gene is fragmented on multiple vectors, with shared restriction enzyme sequences or recombination site sequences connecting the gene fragments, wherein the vectors are used to exclude vectors that do not contain the DNA fragment of interest. The vectors are designed and arranged such that an undesired recombination event or ligation event will result in the creation of an intact Tse2 gene on the undesired plasmid, thus resulting in the death of the cells containing the undesired vector with the functional Tse2 gene. The vectors containing the intact Tse2 gene also are lacking the DNA fragment of interest, and are thus excluded from selection.

### REFERENCES

Abdallah, A. M., Gey van Pittius, N. C., Champion, P. A., Cox, J., Luirink, J., Vandenbroucke-Grauls, C. M., Appelmelk, B. J., and Bitter, W. (2007). Type VII secretion—mycobacteria show the way. Nat Rev Microbiol 5, 883-891.
Arnoldo, A., Curak, J., Kittanakom, S., Chevelev, I., Lee, V. T., Sahebol-Amri, M., Koscik, B., Ljuma, L., Roy, P. J., Bedalov, A., et al. (2008). Identification of small molecule inhibitors of Pseudomonas aeruginosa exoenzyme S using a yeast phenotypic screen. PLoS genetics 4, e1000005.
Aschtgen, M. S., Bernard, C. S., De Bentzmann, S., Lloubes, R., and Cascales, E. (2008). SciN is an outer membrane lipoprotein required for Type VI secretion in enteroaggregative Escherichia coli. J Bacteriol.
Ballister, E. R., Lai, A. H., Zuckermann, R. N., Cheng, Y., and Mougous, J. D. (2008). In Vitro Self-Assembly of Tailorable Nanotubes from a Simple Protein Building Block. Proc Natl Acad Sci USA 105, 3733-3738.
Baynham, P. J., Ramsey, D. M., Gvozdyev, B. V., Cordonnier, E. M., and Wozniak, D. J. (2006). The Pseudomonas aeruginosa ribbon-helix-helix DNA-binding protein AlgZ (AmrZ) controls twitching motility and biogenesis of type IV pili. J Bacteriol 188, 132-140.
Bingle, L. E., Bailey, C. M., and Pallen, M. J. (2008). Type VI secretion: a beginner's guide. Current opinion in microbiology 11, 3-8.
Bladergroen, M. R., Badelt, K., and Spaink, H. P. (2003). Infection-blocking genes of a symbiotic Rhizobium leguminosarum strain that are involved in temperature-dependent protein secretion. Mol Plant Microbe Interact 16, 53-64.
Blondel, C. J., Jimenez, J. C., Contreras, I., and Santiviago, C. A. (2009). Comparative genomic analysis uncovers 3 novel loci encoding type six secretion systems differentially distributed in Salmonella serotypes. BMC genomics 10, 354.
Boyer, F., Fichant, G., Berthod, J., Vandenbrouck, Y., and Attree, I. (2009). Dissecting the bacterial type VI secretion system by a genome wide in silico analysis: what can be learned from available microbial genomic resources? BMC genomics 10, 104.
Brencic, A., and Lory, S. (2009). Determination of the regulon and identification of novel mRNA targets of Pseudomonas aeruginosa RsmA. Mol Microbiol 72, 612-632.
Burns, D. L. (2003). Type IV transporters of pathogenic bacteria. Current opinion in microbiology 6, 29-34.
Cambronne, E. D., and Roy, C. R. (2006). Recognition and delivery of effector proteins into eukaryotic cells by bacterial secretion systems. Traffic (Copenhagen, Denmark) 7, 929-939.
Cardona, S. T., and Valvano, M. A. (2005). An expression vector containing a rhamnose-inducible promoter provides tightly regulated gene expression in Burkholderia cenocepacia. Plasmid 54, 219-228.
Christie, P. J., Atmakuri, K., Krishnamoorthy, V., Jakubowski, S., and Cascales, E. (2005). Biogenesis, architecture, and function of bacterial type iv secretion systems. Annual review of microbiology 59, 451-485.
D'Argenio, D. A., Wu, M., Hoffman, L. R., Kulasekara, H. D., Deziel, E., Smith, E. E., Nguyen, H., Ernst, R. K., Larson Freeman, T. J., Spencer, D. H., et al. (2007). Growth phenotypes of Pseudomonas aeruginosa lasR mutants adapted to the airways of cystic fibrosis patients. Mol Microbiol 64, 512-533.
Das, S., Chakrabortty, A., Banerjee, R., and Chaudhuri, K. (2002). Involvement of in vivo induced icmF gene of Vibrio cholerae in motility, adherence to epithelial cells, and conjugation frequency. Biochem Biophys Res Commun 295, 922-928.
Deretic, V., Schurr, M. J., and Yu, H. (1995). Pseudomonas aeruginosa, mucoidy and the chronic infection phenotype in cystic fibrosis. Trends Microbiol 3, 351-356.
Emanuelsson, O., Brunak, S., von Heijne, G., and Nielsen, H. (2007). Locating proteins in the cell using TargetP, SignalP and related tools. Nature protocols 2, 953-971.
Eng, J. K., McCormack, A. L., and Yates, J. R. (1994). An approach to correlate tandem mass-spectral data of peptides with amino-acid sequences in a protein database. J Am Soc Mass Spectrom 5, 976-989.
Enos-Berlage, J. L., Guvener, Z. T., Keenan, C. E., and McCarter, L. L. (2005). Genetic determinants of biofilm development of opaque and translucent Vibrio parahaemolyticus. Mol Microbiol 55, 1160-1182.
Filloux, A. (2009). The type VI secretion system: a tubular story. The EMBO journal 28, 309-310.
Foundation, C. F. (2007). Cystic Fibrosis Foundation Patient Registry, (Cystic Fibrosis Found, Bethesda, Md.)
Garcia, J. T., Ferracci, F., Jackson, M. W., Joseph, S. S., Pattis, I., Plano, L. R., Fischer, W., and Plano, G. V. (2006). Measurement of effector protein injection by type III and type IV secretion systems by using a 13-residue phosphorylatable glycogen synthase kinase tag. Infection and immunity 74, 5645-5657.
Gerdes, K., Christensen, S. K., and Lobner-Olesen, A. (2005). Prokaryotic toxin-antitoxin stress response loci. Nat Rev Microbiol 3, 371-382.
Gibbs, K. A., Urbanowski, M. L., and Greenberg, E. P. (2008). Genetic determinants of self identity and social recognition in bacteria. Science 321, 256-259.
Goodman, A. L., Kulasekara, B., Rietsch, A., Boyd, D., Smith, R. S., and Lory, S. (2004). A signaling network reciprocally regulates genes associated with acute infection and chronic persistence in Pseudomonas aeruginosa. Dev Cell 7, 745-754.
Hoffman, L. R., Deziel, E., D'Argenio, D. A., Lepine, F., Emerson, J., McNamara, S., Gibson, R. L., Ramsey, B. W., and Miller, S. I. (2006). Selection for Staphylococcus aureus small-colony variants due to growth in the presence of Pseudomonas aeruginosa. Proc Natl Acad Sci USA 103, 19890-19895.
Hsu, F., Schwarz, S., and Mougous, J. D. (2009). TagR promotes PpkA-catalysed type VI secretion activation in Pseudomonas aeruginosa. Mol Microbiol 72, 1111-1125.
Jacobs, M. A., Alwood, A., Thaipisuttikul, I., Spencer, D., Haugen, E., Ernst, S., Will, O., Kaul, R., Raymond, C., Levy, R., et al. (2003). Comprehensive transposon mutant library of Pseudomonas aeruginosa. Proc Natl Acad Sci USA 100, 14339-14344.
Keller, A., Nesvizhskii, A. I., Kolker, E., and Aebersold, R. (2002). Empirical statistical model to estimate the accuracy of peptide identifications made by MS/MS and database search. Analytical chemistry 74, 5383-5392.
Kessler, E., Safrin, M., Olson, J. C., and Ohman, D. E. (1993). Secreted LasA of Pseudomonas aeruginosa is a staphylolytic protease. The Journal of biological chemistry 268, 7503-7508.
Lapouge, K., Schubert, M., Allain, F. H., and Haas, D. (2008). Gac/Rsm signal transduction pathway of gamma-proteobacteria: from RNA recognition to regulation of social behaviour. Mol Microbiol 67, 241-253.
Laskowski, M. A., and Kazmierczak, B. I. (2006). Mutational analysis of RetS, an unusual sensor kinase-response regulator hybrid required for Pseudomonas aeruginosa virulence. Infection and immunity 74, 4462-4473.
Laskowski, M. A., Osborn, E., and Kazmierczak, B. I. (2004). A novel sensor kinase-response regulator hybrid regulates type III secretion and is required for virulence in Pseudomonas aeruginosa. Mol Microbiol 54, 1090-1103.
Lawley, T. D., Klimke, W. A., Gubbins, M. J., and Frost, L. S. (2003). F factor conjugation is a true type IV secretion system. FEMS Microbiol Lett 224, 1-15.
Leiman, P. G., Basler, M., Ramagopal, U. A., Bonanno, J. B., Sauder, J. M., Pukatzki, S., Burley, S. K., Almo, S. C., and Mekalanos, J. J. (2009). Type VI secretion apparatus and phage tail-associated protein complexes share a common evolutionary origin. Proc Natl Acad Sci USA 106, 4154-4159.
Liu, H., Sadygov, R. G., and Yates, J. R., 3rd (2004). A model for random sampling and estimation of relative protein abundance in shotgun proteomics. Analytical chemistry 76, 4193-4201.
Luo, Z. Q., and Isberg, R. R. (2004). Multiple substrates of the Legionella pneumophila Dot/Icm system identified by interbacterial protein transfer. Proc Natl Acad Sci USA 101, 841-846.
Ma, A. T., McAuley, S., Pukatzki, S., and Mekalanos, J. J. (2009). Translocation of a Vibrio cholerae type VI secretion effector requires bacterial endocytosis by host cells. Cell host & microbe 5, 234-243.
Mougous, J. D., Cuff, M. E., Raunser, S., Shen, A., Zhou, M., Gifford, C. A., Goodman, A. L., Joachimiak, G., Ordonez, C. L., Lory, S., et al. (2006). A virulence locus of Pseudomonas aeruginosa encodes a protein secretion apparatus. Science 312, 1526-1530.
Mougous, J. D., Gifford, C. A., Ramsdell, T. L., and Mekalanos, J. J. (2007). Threonine phosphorylation post-translationally regulates protein secretion in Pseudomonas aeruginosa. Nature cell biology 9, 797-803.
Mumberg, D., Muller, R., and Funk, M. (1995). Yeast vectors for the controlled expression of heterologous proteins in different genetic backgrounds. Gene 156, 119-122.
Nguyen, D., and Singh, P. K. (2006). Evolving stealth: genetic adaptation of Pseudomonas aeruginosa during cystic fibrosis infections. Proc Natl Acad Sci USA 103, 8305-8306.
Palmer, K. L., Aye, L. M., and Whiteley, M. (2007). Nutritional cues control Pseudomonas aeruginosa multicellular behavior in cystic fibrosis sputum. J Bacteriol 189, 8079-8087.
Pell, L. G., Kanelis, V., Donaldson, L. W., Howell, P. L., and Davidson, A. R. (2009). The phage lambda major tail protein structure reveals a common evolution for long-tailed phages and the type VI bacterial secretion system. Proc Natl Acad Sci USA 106, 4160-4165.
Peubez, et al. Microbial Cell Factories 2010, 9:65
Potvin, E., Lehoux, D. E., Kukavica-Ibrulj, I., Richard, K. L., Sanschagrin, F., Lau, G. W., and Levesque, R. C. (2003). In vivo functional genomics of Pseudomonas aeruginosa for high-throughput screening of new virulence factors and antibacterial targets. Environmental microbiology 5, 1294-1308.
Pukatzki, S., Ma, A. T., Revel, A. T., Sturtevant, D., and Mekalanos, J. J. (2007). Type VI secretion system translocates a phage tail spike-like protein into target cells where it cross-links actin. Proc Natl Acad Sci USA 104, 15508-15513.
Pukatzki, S., McAuley, S. B., and Miyata, S. T. (2009). The type VI secretion system: translocation of effectors and effector-domains. Current opinion in microbiology 12, 11-17.
Rietsch, A., Vallet-Gely, I., Dove, S. L., and Mekalanos, J. J. (2005). ExsE, a secreted regulator of type III secretion genes in Pseudomonas aeruginosa. Proc Natl Acad Sci USA 102, 8006-8011.
Riley, M. A., and Wertz, J. E. (2002). Bacteriocins: evolution, ecology, and application. Annual review of microbiology 56, 117-137.
Ryder, C., Byrd, M., and Wozniak, D. J. (2007). Role of polysaccharides in Pseudomonas aeruginosa biofilm development. Current opinion in microbiology 10, 644-648.
Satchell, K. J. (2009). Bacterial martyrdom: phagocytes disabled by type VI secretion after engulfing bacteria. Cell host & microbe 5, 213-214.
Sibley, C. D., Rabin, H., and Surette, M. G. (2006). Cystic fibrosis: a polymicrobial infectious disease. Future microbiology 1, 53-61.
Singh, P. K., Schaefer, A. L., Parsek, M. R., Moninger, T. O., Welsh, M. J., and Greenberg, E. P. (2000). Quorum-sensing signals indicate that cystic fibrosis lungs are infected with bacterial biofilms. Nature 407, 762-764.
Starkey, M., Hickman, J. H., Ma, L., Zhang, N., De Long, S., Hinz, A., Palacios, S., Manoil, C., Kirisits, M. J., Starner, T. D., et al. (2009). Pseudomonas aeruginosa rugose small-colony variants have adaptations that likely promote persistence in the cystic fibrosis lung. J Bacteriol 191, 3492-3503.
Stover, C. K., Pham, X. Q., Erwin, A. L., Mizoguchi, S. D., Warrener, P., Hickey, M. J., Brinkman, F. S., Hufnagle, W. O., Kowalik, D. J., Lagrou, M., et al. (2000). Complete genome sequence of Pseudomonas aeruginosa PAO1, an opportunistic pathogen. Nature 406, 959-964.
Suarez, G., Sierra, J. C., Erova, T. E., Sha, J., Horneman, A. J., and Chopra, A. K. (2009). A type VI secretion system effector protein VgrG1 from Aeromonas hydrophila that induces host cell toxicity by ADP-ribosylation of actin. J Bacteriol.
Vance, R. E., Rietsch, A., and Mekalanos, J. J. (2005). Role of the type III secreted exoenzymes S, T, and Y in systemic spread of Pseudomonas aeruginosa PAO1 in vivo. Infection and immunity 73, 1706-1713.
Ventre, I., Goodman, A. L., Vallet-Gely, I., Vasseur, P., Soscia, C., Molin, S., Bleves, S., Lazdunski, A., Lory, S., and Filloux, A. (2006). Multiple sensors control reciprocal expression of Pseudomonas aeruginosa regulatory RNA and virulence genes. Proc Natl Acad Sci USA 103, 171-176.
Voggu, L., Schlag, S., Biswas, R., Rosenstein, R., Rausch, C., and Gotz, F. (2006). Microevolution of cytochrome bd oxidase in Staphylococci and its implication in resistance to respiratory toxins released by Pseudomonas. J Bacteriol 188, 8079-8086.
Wargo, M. J., and Hogan, D. A. (2006). Fungal—bacterial interactions: a mixed bag of mingling microbes. Current opinion in microbiology 9, 359-364.
Weber, B., Hasic, M., Chen, C., Wai, S. N., and Milton, D. L. (2009). Type VI secretion modulates quorum sensing and stress response in Vibrio anguillarum. Environmental microbiology.
Wehmhoner, D., Haussler, S., Tummler, B., Jansch, L., Bredenbruch, F., Wehland, J., and Steinmetz, I. (2003). Inter- and intraclonal diversity of the Pseudomonas aeruginosa proteome manifests within the secretome. J Bacteriol 185, 5807-5814.
Yahr, T. L. (2006). A critical new pathway for toxin secretion? N Engl J Med 355, 1171-1172.
Zheng, J., and Leung, K. Y. (2007). Dissection of a type VI secretion system in Edwardsiella tarda. Mol Microbiol 66, 1192-1206.
Zolfaghar, I., Angus, A. A., Kang, P. J., To, A., Evans, D. J., and Fleiszig, S. M. (2005). Mutation of retS, encoding a putative hybrid two-component regulatory protein in Pseudomonas aeruginosa, attenuates multiple virulence mechanisms. Microbes Infect 7, 1305-1316.
Zolfaghar, I., Evans, D. J., Ronaghi, R., and Fleiszig, S. M. (2006). Type III secretion-dependent modulation of innate immunity as one of multiple factors regulated by Pseudomonas aeruginosa RetS. Infection and immunity 74, 3880-3889.

1. A recombinant vector, comprising a first gene coding for type VI secretion exported protein 2 (Tse2), wherein the first gene is operatively linked to a heterologous regulatory sequence.

2. The recombinant vector of claim 1, wherein the vector comprises one or more unique restriction enzyme recognition sites, and wherein cloning of a nucleic acid insert into the one or more unique restriction enzyme recognition sites disrupts expression of the first gene.

3. The recombinant vector of claim 1, wherein the recombinant vector comprises at least a first and a second recombination site flanking a first gene coding for Tse2 operatively linked to a regulatory sequence, wherein said first and second recombination sites do not recombine with each other.

4. The recombinant vector of claim 1, wherein the first gene comprises a nucleotide sequence that can encode an amino acid sequence according to SEQ ID NO:2, SEQ ID NO:4, SEQ ID NO:5, SEQ ID NO:6, SEQ ID NO:7, or SEQ ID NO:8.

5. The recombinant vector of claim 1, wherein the regulatory sequence for the first gene is conditionally controlled.

6. The recombinant vector of claim 1, further comprising a second gene encoding a Tse2 antidote operatively linked to a regulatory sequence.

7. The recombinant vector of claim 6, wherein the second gene encodes type VI secretion immunity protein 2 (Tsi2).

8. The recombinant vector of claim 4, wherein the second gene comprises a nucleotide sequence that can encode an amino acid sequence according to SEQ ID NO:10, SEQ ID NO:11, or SEQ ID NO:13.

9. The recombinant vector of claim 4, wherein the regulatory sequence for the second gene are conditionally controlled.

10. The recombinant vector of claim 1, wherein the vector comprises a plasmid.

11. The recombinant vector of claim 1, wherein the vector comprises a viral nucleic acid.

12. A recombinant host cell comprising the recombinant vector of claim 1.

13. The recombinant host cell of claim 12, wherein the host cell is a prokaryotic cell.

14. The recombinant host cell of claim 12, wherein the host cell is a eukaryotic cell.

15. A method for selectable cloning, comprising culturing the recombinant host cell of claim 12 under conditions suitable for expression of Tse2 from the recombinant vector if no insert is present, and selecting those cells that grow as comprising recombinant vectors with the insert cloned into the expression vector.

16. A method for production of a cloning vector that lacks an insert, comprising culturing the recombinant host cell of claim 12 under conditions suitable for vector replication and expression of Tse2, wherein the recombinant host cells further express a Tse2 antidote, and isolating vector from the host cells.

17. The method of claim 16, wherein the Tse2 antidote comprises Tsi2.

18. The method of claim 17, wherein the Tsi2 comprises an amino acid sequence according to SEQ ID NO:10, SEQ ID NO:11, or SEQ ID NO:13.

19. A recombinant vector, comprising a nucleic acid encoding Tsi2, wherein the nucleic acid is operatively linked to a regulatory sequence.

20. The recombinant vector of claim 19, wherein the nucleic acid comprises a nucleotide sequence that can encode an amino acid sequence according to SEQ ID NO:10, SEQ ID NO:11, or SEQ ID NO:13.

21. A host cell comprising in its genome, a first recombinant gene coding for Tse2 operatively linked to a regulatory sequence.

22. The host cell of claim 21, further comprising a second recombinant gene coding for an antidote for Tse2, wherein the second gene is operatively linked to a regulatory sequence.

23. The host cell of claim 21, wherein the first recombinant gene and/or the second recombinant gene are present as a chromosomal insertion.

24. The host cell of claim 22, wherein the second gene codes for type Tsi2.

25. The host cell of claim 21, wherein the regulatory sequence for the first gene are conditionally controlled.

26. The host cell of claim 21, wherein the regulatory sequence for the second gene are conditionally controlled.

27. The host cell of claim 21, wherein the host cell is a bacterial cell.

28. The host cell of claim 21, wherein the first gene comprises a nucleotide sequence that can encode an amino acid sequence according to SEQ ID NO:2, SEQ ID NO:4, SEQ ID NO:5, SEQ ID NO:6, SEQ ID NO:7, or SEQ ID NO:8.

29. The host cell of claim 22, wherein the second gene comprises a nucleotide sequence that can encode an amino acid sequence according to SEQ ID NO:10, SEQ ID NO:11, or SEQ ID NO:13.

30. The vector of claim 1, wherein the vector is linear.

31. The vector of claim 1, wherein the vector is treated to reduce the formation of vectors which lack the DNA fragment of interest.

32. The vector of claim 1, wherein the vector is suitable for TA or topoisomerase cloning reactions.
